(19) United States
(12) Patent Application Publication
(10) Pub. No.: US 2012/0040335 A1
(43) Pub. Date: Feb. 16, 2012

(54) Identification of Porcine Reproductive and Respiratory Syndrome Virus

(75) Inventors: Ying Fang, Brookings, SD (US); Elizabeth M. Brown, Pierre, SD (US); Craig Welbon, Wentworth, SD (US); Eric A. Nelson, Volga, SD (US)

(73) Assignee: SOUTH DAKOTA STATE UNIVERSITY, Brookings, SD (US)

(21) Appl. No.: 13/131,592

(22) PCT Filed: Nov. 25, 2009
(86) PCT No.: PCT/US09/06289

§ 371 Date: Oct. 16, 2011

Related U.S. Application Data
(60) Provisional application No. 61/200,398, filed on Nov. 26, 2008.

(51) Int. Cl. C12Q 1/70

(52) U.S. Cl. 435/5

(57) ABSTRACT

An enzyme-linked immunosorbent assay (ELISA) is based on the non-structural protein 7 (nsp7) of porcine reproductive and respiratory syndrome virus (PRRSV) and provides for the simultaneous detection and differentiation of serum antibodies directed against Type 1 (European) and Type 2 (North American) PRRSV. The invention provides a serological assay for the detection and/or differentiation of serum antibodies directed against Type 1 and/or Type 2 PRRSV utilizing PRRSV nsp7 as an antigen, and provides a diagnostic method for the detection of PRRSV infection, epidemiological surveys, and outbreak investigations. The invention may be used either alone or as a follow-up assay to determine the true status of unexpected positive results that may occur using other assays, such as the IDEXX HERDCHEK PRRS ELISA.

### CROSS-REFERENCE TO RELATED APPLICATION

This application claims the benefit of U.S. Provisional Application No. 61/200,398, filed Nov. 26, 2008, the disclosure of which is incorporated herein by reference in its entirety.

### STATEMENT REGARDING FEDERALLY SPONSORED RESEARCH OR DEVELOPMENT

This invention was made with Government support under the National Pork Board grant number #05-155 and the National Research Initiative of the USDA Cooperative State Research, Education and Extension Service grant number 2004-35605-14197 (through PRRSV CAP grant #580). The U.S. Government may have certain rights to this invention.

### TECHNICAL FIELD

The invention relates to biotechnology, more particularly to serological assays for detecting porcine reproductive and respiratory syndrome virus and/or differentiating between genotypes of the virus.

### BACKGROUND

Porcine reproductive and respiratory syndrome (PRRS) continues to be one of the most devastating diseases of swine throughout the world. The etiological agent, (PRRSV) was classified in the genus Arterivirus, family Arteriviridae and order Nidovirales. Nucleotide sequence comparisons show that PRRSV can be divided into distinct European (Type 1) and North American (Type 2) genotypes, possessing only about 63% nucleotide identity at the genomic level (Allende et al., 1999; Nelsen et al., 1995). PRRSV is a small, enveloped virus containing a single positive-stranded RNA genome. The PRRSV genome is about 15 kb in length and contains nine open reading frames. The replicase-associated genes, ORF1a and ORF1b, situated at the 5′ end of the genome, represent nearly 75% of the viral genome. The ORF1a encoded polyprotein is predicted to be cleaved at eight sites to form nine end products: nsp1α, nsp1β, and nsp2 through nsp8. Proteolytic cleavage of the ORF1b portion of the replicase generates products nsp9 though nsp12 (Den Boon et al., 1995; van Dinten et al., 1996). The nonstructural proteins (nsp) derived from ORF1a possess proteolytic activities and are responsible for processing the other nsp cleavage products. ORF1b cleavage products are involved in virus transcription and replication (Gorbalenya et al., 1989; den Boon et al., 1991; Godeny et al., 1993; van Dinten et al., 1996; Gorbalenya et al., 1989; Snijder and Meulenberg, 1998). The 3′ end of the genome encodes four membrane-associated glycoproteins (GP2a, GP3, GP4 and GP5; encoded by sg mRNAs 2-5), two unglycosylated membrane proteins (2b and M; encoded by sg mRNAs 2 and 6), and a nucleocapsid protein (N; encoded by sg mRNA 7) (Meulenberg et al., 1995, 1996; Meng et al., 1995; Mounir et al., 1995; Bautista et al., 1996; Mardassi et al., 1996; Wu et al., 2001, 2005).

In the absence of effective vaccines and therapeutic drugs, one of the key approaches to achieve the “National PRRS Elimination” is to identify PRRSV infected pigs, so that such pigs can be quarantined, isolated or removed from herds to block or reduce the transmission of infection to susceptible animals Serological testing to determine the PRRS status of herds and individual animals is often included in management strategies for monitoring and controlling PRRS. A large body of information indicates that the nucleocapsid (N) protein is the most immunogenic protein and an ideal target for serological assays that can be used for the identification of infected pigs (Ferrin et al., 2004; Seuberlich et al., 2002; Wootton et al., 1998).

Currently, the IDEXX HerdChek® PRRS 2XR ELISA, which is based on N protein as the antigen, is widely used for the detection of antibodies to either North American Type 2 or European-like Type 1 PRRSV. Nevertheless, individual unexpected positive IDEXX ELISA results in otherwise seronegative herds have caused great concern. These false positives require the use of alternative antigens as a more accurate indicator of infection. Previous studies have shown that certain non-structural proteins, such as nsp2 are also highly immunogenic (Fang et al., 2004; Oleksiewicz et al., 2001a, b, 2002; Johnson et al., 2007). However, nsp 2 is generally insoluble, making it difficult to work with as the base antigen in an ELISA.

The present invention provides reagents and/or methods that allow for the identification of a humoral immune response to PRRSV-infected pigs using a non-structural protein.

### SUMMARY OF THE INVENTION

The invention provides a method that is useful for veterinary diagnostic laboratories and researchers. The invention relates to an enzyme-linked immunosorbent assay (ELISA) that is based on the non-structural protein 7 (nsp7) of porcine reproductive and respiratory syndrome virus (PRRSV). The invention provides materials and methods that also allow for the simultaneous detection and differentiation of serum antibodies directed against Type 1 (European) and Type 2 (North American) PRRSV.

The invention further relates to a serological assay for the detection and/or differentiation of serum antibodies directed against Type 1 and/or Type 2 PRRSV utilizing PRRSV nsp7 as an antigen. The invention further relates to a diagnostic method for the detection of PRRSV infection, epidemiological surveys, and outbreak investigations. The invention may be used either alone or as a follow-up assay to determine the true status of unexpected positive results that may occur using other assays, such as, for example, the IDEXX HERDCHEK PRRS ELISA.

The assay is convenient with respect to antigen production and is also reliable, economical, and highly sensitive and serotype specific.

The nsp7-based ELISA of the invention provides an improvement over other non-structural protein-based ELISAs, due at least in part to the ease of antigen preparation and the high specificity and accuracy of the diagnostic test application.

In an exemplary embodiment, recombinant nsp 7 protein, or fragments thereof, is used as an antigen in an in vitro dual enzyme-linked immunosorbent assay (nsp7 Dual-ELISA) for the simultaneous detection and differentiation of serum antibodies directed against Type 1 and Type 2 PRRSV.

The invention also relates to kits for the detection of antibodies directed to nsp7 or a fragment thereof.

### BRIEF DESCRIPTION OF THE DRAWINGS

FIG. 1 depicts a Sodium dodecyl sulfate-polyacrylamide (SDS-PAGE) gel electrophoresis of recombinant PRRSV nonstructural protein preparations followed by Coomassie blue staining. Lane 1 shows the protein molecular size standard; Lanes 2 through 6 represents nsp1, nsp2, nsp4, nsp7 and nsp8 preparations, respectively. NA═North American genotype (Type II); EU=European genotype (Type I). Note: nsp1 is further cleaved into nsp1α and nsp1β subunits (5, 13). Intact nsp1 and 26 kDa nsp1β eluted from the immobilized metal-affinity column were shown in lane 2.

FIG. 2 depictsthe kinetics of antibody response to PRRSV nsps. Pigs were experimentally infected with Type II PRRSV, VR2332. The serum samples were from 0 to 202 dpi as indicated. For the nsp4 and nsp8, serum samples from 10 pigs were tested; for the nsp1, nsp2 and nsp7, serum samples from 30 pigs were tested.

FIG. 3 depicts a two-graph ROC plot of the PRRSV nsp7-based ELISA. The graphs were calculated using 965 (Type I nsp7) and 1,726 (Type II nsp7) individual animal serum samples and GraphROC software. The downward-pointing histogram on the left side of the figure represents the uninfected animals, and the upward-pointing histogram on the right side of the figure represents the PRRSV-infected animals. The green line represents the diagnostic sensitivity for the assay as the cutoff S/P ratio is moving from 0 to 2.7. The red line represents the diagnostic specificity for the assay as the cutoff S/P ratio is moving from 0 to 2.7. The black dashed vertical line represents the optimized cutoff value of 0.51 (Type I, FIG. 3A) and 0.52 (Type II, FIG. 3B), which corresponds to the maximum diagnostic sensitivity and specificity.

FIG. 4 depicts the differentiation of Type I and Type II PRRSV using the nsp7 dual-ELISA. The distribution of individual samples with S/P value above cutoff in the Type I or Type II nsp7 ELISA is shown according to the calculated r values. The percentage of sera samples comparing to the total number of positive sera in each test is shown in the vertical axis. For each positive sample, an r value, representing the log 10 of the ratio obtained by dividing the S/P ratio observed in the Type I nsp7 ELISA by the S/P ratio observed in the Type II nsp7 ELISA, was calculated. Thus, r values of >0 represent positives in the Type I nsp7 ELISA, and r values of <0 represent positives in the Type II nsp7 ELISA.

### DETAILED DESCRIPTION OF THE INVENTION

As used herein, “Antibody” means naturally occurring antibodies as well as non-naturally occurring antibodies, including, for example, single chain antibodies, chimeric, bifunctional and humanized antibodies, as well as antigen-binding fragments thereof (see Huse et al., Science 246:1275-1281, 1989; Winter and Harris, Immunol. Today 14:243-246, 1993; Ward et al., Nature 341:544-546, 1989; Harlow and Lane, Antibodies: A laboratory manual (Cold Spring Harbor Laboratory Press, 1999); and Hilyard et al., Protein Engineering: A practical approach (IRL Press 1992); Borrabeck, Antibody Engineering, 2d ed. (Oxford Univ. Press 1995).

As used herein, “about” means reasonably close to, or approximately, a little more or less than the stated number or amount.

As used herein, “serum” means whole blood or any fraction thereof, for example plasma, platelets, and a plasma concentrate.

As used herein, “detectable moiety” or a “label” refers to a compound or composition that is detectable at a low concentration by spectroscopic, photochemical, biochemical, immunochemical, or chemical means. For example, useful labels include, but are not limited to, ³²P, ³⁵S, ³H, ¹²⁵I, fluorescent dyes, electron-dense reagents, enzymes, magnetic particles, biotin-streptavadin, dioxigenin, haptens and proteins.

As used herein, “diagnosis” or “diagnostic” means a prediction of the type of disease or condition from a set of marker values, such as the presence or absence of an immune response to PRRSV.

As used herein, “ELISA” means enzyme-linked immunosorbent assay, including direct and ligand-capture ELISAs, along with radioimmunoassays (RIAs). See U.S. Pat. Nos. 5,192,660 and 4,474,892, along with International Patent Publications WO 2008/060777, WO 2007/066231, WO 2007/008966, WO 2006/009880 and WO 1990/003447.

As used herein, “Enzyme” means a protein or ordered aggregate of proteins that catalyzes a specific biochemical reaction, wherein the enzyme is not itself altered in the process.

As used herein, “PCR” means polymerase chain reaction.

The “percent (%) sequence identity” between two polypeptide sequences can be determined according to methods known in the art, including, but not limited to, the BLAST program (Basic Local Alignment Search Tool, Altschul and Gish (1996) Meth Enzymol 266: 460-480; Altschul (1990) J Mol Biol 215: 403-410)).

As used herein a “Substantially Identical” polypeptide sequence means an amino acid sequence which differs from a reference sequence only by conservative amino acid substitutions, for example, substitution of one amino acid for another of the same class (for example, valine for glycine, arginine for lysine, etc.) or by one or more non-conservative substitutions, deletions, or insertions located at positions of the amino acid sequence which do not destroy the function of the polypeptide (for example, its ability to be recognized by antibodies as described herein). Preferably, such a sequence is at least 50-70%, more preferably at least 70-85%, and most preferably at least 85-99% substantially identical at the amino acid level to the sequence used for comparison. Two sequences, either nucleic acids or proteins, can be compared using any of the commercially available computer algorithms routinely used to produce sequence alignments. For example, to find the alignment of two or more sequences the parameters in the program may be set to maximizes the number of matches and minimizes the number of gaps.

As used herein “Positioned for Expression” means that the nucleic acid molecule is operably linked to a sequence which directs transcription and translation of a nucleic acid molecule encoding a desired protein or peptide sequence.

As used herein, “nsp7” means any porcine arteritis virus non-structural protein 7 (nsp7), or fragment thereof, as defined by its location in open reading frame 1a (ORF1a) and identification as nsp7, such as those sequences that can be found in GenBank™, including, but not limited to, accession numbers X53459, M96262, U15146, AY588319, AY457635, Q9YN02, Q8B912, Q9WJB2, NP740601, NPO66135, NC001961 and U63121. Full length Nsp7 is approximately 259 amino acids and is cleaved from the translation product of ORF1a. The term “nsp7” also includes purified proteins or fragments thereof, which may or may not be modified or deliberately engineered (see U.S. Pat. No. 7,169,758). For example, modifications in a nsp7 peptide or DNA sequences can be made by those skilled in the art using known techniques and include, but are not limited to, amino acid alteration, substitution, replacement, insertion or deletion. Preferably, such alteration, substitution, replacement, insertion or deletion retains the desired antigenic epitope of the nsp7 protein.

As used herein, “EU-nsp7” means the recombinant protein deriving from Type 1 PRRSV (i.e., the European genotype), and as used herein, “NA-nsp7” means the recombinant protein deriving from Type 2 PRRSV (i.e., the North American genotype).

As used herein, “Peptide,” “Polypeptide” and “Protein” include polymers of five or more amino acids joined by peptide bonds, and includes post-translational-modification and amino acid analogues. No distinction, based on length, is intended between a peptide, a polypeptide or a protein.

As used herein, “sample” means any sample of biological material derived from a subject, such as, but not limited to, blood, plasma, and other fluids, which has been removed from the body of the subject and contains or is thought to contain antibodies produced by the subject. The sample which is tested according to the method of the invention may be tested directly or indirectly and may require some form of treatment prior to testing. For example, a blood sample may require one or more separation steps prior to testing. Further, the sample may require the addition of a reagent, such as a buffer.

As used herein, “subject” means a mammal, including, but not limited to, a porcine animal.

As used herein, “treating” or “treatment” does not require a complete cure. It means that the symptoms of the underlying disease are at least reduced, and/or that one or more of the underlying cellular, physiological, or biochemical causes or mechanisms causing the symptoms are reduced and/or eliminated. It is understood that reduced, as used in this context, means relative to the state of the disease, including the molecular state of the disease, not just the physiological state of the disease.

As used herein, “comprising,” “including,” “containing,” “characterized by,” and grammatical equivalents thereof are inclusive or open-ended terms that do not exclude additional, unrecited elements or method steps, but also includes the more restrictive terms “consisting of” and “consisting essentially of.”

As used herein and in the appended claims, the singular forms, for example, “a”, “an”, and “the,” include the plural, unless the context clearly dictates otherwise. For example, reference to “an epitope of nsp7” may include a plurality of such epitopes.

The invention includes kits, for example, an immunoassay kit for detecting antibodies to PRRSV in a biological sample. A kit of the invention may comprise: (a) a capture reagent, such as a recombinant nsp7 protein or fragment thereof; and (b) a detection reagent, such as a detectable antibody or labeled antibody that binds to porcine antibodies (see U.S. Pat. No. 7,449,296 and International Patent Publication WO 96/06619). In certain embodiments, the kit further comprises a solid support for the capture reagents. For example, the capture reagents can be immobilized on the solid support (e.g., a microtiter plate). In certain embodiments, the kit further comprises a detection means (e.g., colormetric means, fluorimetric means, etc.) for the detectable antibodies. In certain embodiments, the kit further comprises instructions. In certain embodiments, the kit further comprises standards against which samples may be measured.

In an exemplary embodiment, a device of the invention is a test strip. Such a test strip may be designed to operate solely based on a liquid available from a biological sample applied thereto (see for example U.S. Pat. Nos. 5,591,645 and 4,235,601). Alternatively, the test strip may be designed to operate in connection with a detection system or developing solution that detects the presence of an antibody in the biological sample. In another exemplary embodiment, the test strip may be embodied in a housing or casing.

While the invention is described in terms of an ELISA where the capture molecule is nsp7 or a fragment thereof, it will be understood in light of the disclosure herein that the capture molecule may also be an antibody (either monoclonal or polyclonal) that recognizes nsp7 or a fragment thereof, with concomitant changes in the method steps disclosed herein.

The current study aimed to determine the humoral immune response to the PRRSV nonstructural proteins and to develop new tools for identification of PRRSV infected animals. Previous studies of the humoral immune response to PRRSV have focused mainly on detection of antibodies to viral structural proteins, especially nucleocapsid. Several studies showed that certain nonstructural proteins, such as nsp1 and nsp2 are highly immunogenic. Antibody responses to linear epitopes in nsp2 have been reported to appear within 1-4 weeks of infection in Type I and Type II PRRSV strains. Johnson et al. observed robust and rapid cross-reactive antibody responses induced by nsp1 and nsp2 to vaccine and field isolates, and substantially higher levels of immunoreactivity related to conformational epitopes. In this study, our data demonstrated that nsp7 is also highly immunogenic. Analysis of the kinetics of antibody response showed that response to nsp7 is comparable to antibody response to nsp1 and nsp2 as well as antigens used in the commercial IDEXX ELISA. As indicated by Johnson et al., nsps are available from the earliest time of infection for presentation to the immune system in the context of major histocompatibility complex (MHC) class I antigen-presentation pathways. As cytolytic infection also releases viral proteins into interstitial spaces, it is hypothesized that a pronounced antibody response, equivalent to the immune response to structural proteins, would be generated to nonstructural proteins. One intriguing feature of the antibody response to nsp antigens was the sustained antibody titers over a 202 day period of infection, while the antibody response to IDEXX antigen, N protein, showed a gradual decay in titers after 126 dpi. The mechanism for sustained levels of nsp antigen may reflect the long-term retention and presentation of nsp to the immune system.

To select an antigen for diagnostic test development, we compared the correlation between the PRRSV nsp ELISA with IDEXX ELISA. Our results showed that nsp2 and nsp7-based ELISA had higher correlation with those of the IDEXX ELISA. We further compared the amino acid sequences of nsp2 and nsp7. Our previous studies showed that the PRRSV nsp2 region is highly variable within and between genotypes with 70.6%-91.6% amino acid identity within Type I PRRSV and 74.9%-95.6% amino acid identity within Type II PRRSV, but only 33.8% identity between Type I and Type II genotypes. The central region of the nsp2 contains hypervariable domains with insertions and deletions, and most identified B-cell epitopes are located in these regions. In contrast, the nsp7 is relatively conserved within each genotype and is divergent between genotypes Amino acid sequence comparisons showed that nsp7 shares 96.7%-97.4% amino acid identity within Type I PRRSV and 84.9%-100% amino acid identity within Type II PRRSV, but only about 45% identity between Type I and Type II genotypes. These results suggest that the nsp7-based ELISA could be able to detect genotype specific anti-nsp7 antibody responses. Shown below sequentially is an amino acid alignment of Type 1 PRRSV nsp2 and the amino acid alignment of Type 2 PRRSV nsp2. The nsp2 cleavage product is based on the predicted cleavage of the ORF1a polyprotein (Snijder et al., 1995; van Dinten et al., 1996; Allende et al., 1999.). Underlined regions show B cell epitope sites (ES) which, in the Type 1 PRRSV nsp2, were identified by Oleksiewicz et al. (2002) and, in the Type 2 PRRSV nsp2, were identified by de Lima et al. (2006). The box identifies epitopes used for development of differential ELISAs. Asterisks identify deleted amino acids. Therefore, an nsp7-based ELISA was designed as a serology diagnostic assay for detection and differentiation of Type 1 and Type 2 PRRSV.

| AMINO ACID ALIGNMENT OF TYPE 1 PRRSV NSP2 | | | |
|---|---|---|---|
| | 386 + | | |
| Lelystad | AAGKRARAKR AAKSEKDSAP TPKVALPVPT CGITTYSPPT DGSCGWHVLA | SEQ ID NO: 1 | |
| 01-07 | ---------- ------G--- ----V----- --V------- ---------- | SEQ ID NO: 2 | |
| 02-11 | ---------- -V-G------ --E---S--- --T------- ---------- | SEQ ID NO: 3 | |
| 01-08 | ---------- -T-G-----L A-I-P----- ---------- ---------- | SEQ ID NO: 4 | |
| | 436 ES2 | | |
| Lelystad | AIMNRMINGD FTSPLTQYNR PEDDWASDYD LVQAIQCLRL PATVVRNRAC | | |
| 01-07 | ---------- ---------- --------F- -I------Q- ---------- | | |
| 02-11 | ---------- -----APH-- ---------- --------Q- ---------- | | |
| 01-08 | --V------- -----P---- ---------- -A------Q- ---------- | | |
| | 486 + | | |
| Lelystad | PNAKYLIKLN GVHWEVEVRS GMAPRSLSRE CVVGVCSEGC VAPPYPADGL | | |
| 01-07 | ---------- ---------- ---------- ---------- ---------- | | |
| 02-11 | ---------- -------A-- ---------- ---------- ---------- | | |
| 01-08 | ---------- ---------- ---------- ---------- ---------- | | |
| | 536 | | |
| Lelystad | PKRALEALAS AYRLPSDCVS SGIADFLANP PPQEFWTLDK MLTSPSPERS | | |
| 01-07 | ---------- ---------- ---------- ----L----R ---------- | | |
| 02-11 | ---------- ---------- ---------- ---------- ---------- | | |
| 01-08 | ---------- ---------- --------D- ---------- ---------- | | |
| | 586 | | |
| Lelystad | GFSSLYKLLL EVVPQKCGAT EGAFIYAVER MLKDCPSSKQ AMALLAKIKV | | |
| 01-07 | ---------- ----K----- ---------- ---------- --T------- | | |
| 02-11 | ---------- ----R---T- ----T----- ---------- ---------- | | |
| 01-08 | ---------- ---------- ----V----- -------PE- ---------- | | |
| | 636 | | |
| Lelystad | PSSKAPSVSL DECFPTDVLA DFEPASQERP QSSGAAVVLC SPDAKEFEEA | | |
| 01-07 | ---------- --------S- -----F---- ------D--- -LG-----G- | | |
| 02-11 | ---------- -----A-AP- -----F--K- R-----IA-* ****-G-G-- | | |
| 01-08 | ---------- -----AG-P- -----F---- R-P----A-- -----G--GT | | |
| | 686 ES3 | | |
| Lelystad | APEEVQESGH KAVHSALLAE GPNNEQVQVV AGEQLKLGGC GLAVGNAHEG | | |
| 01-07 | --G----G-D E--R--P--V DL-D-HAR-- V--------- --T------- | | |
| 02-11 | -----L--SR -T----*-RA EGS---A-A- -V-------- -S------G- | | |
| 01-08 | -S--A----- ----AVP--- ---------- -----E---- ---I-S-Q** | | |
| | 736 ES4 | | |
| Lelystad | [symbol] SPSDP MKENMLNSRE DEPLDLSQPA PASTTTLVRE | | |
| 01-07 | V-APT-P--- -S-------S -RG------- -------L-- -VA------- | | |
| 02-11 | VP-L------ ----F--P-S --G-T---L- -------R-- L-V--S--K- | | |
| 01-08 | ********** *****-S--S KR---H---- -------H-- --A-----G- | | |
| | 786 | | |
| Lelystad | QTPDNPGSDA GALPVTVREF VPTGPILCHV EHCGTESGDS SSPLDLSDAQ | | |
| 01-07 | ---------- -P-------L A------R-- --------E- ----NC---- | | |
| 02-11 | --SG---LG- ---------- -----A-R-- ----A--D-- ----G---T- | | |
| 01-08 | ---------- S---IA-G-- -------R-- ---------- -------F-- | | |
| | 836 ES5 | | |
| Lelystad | TLDQPLNLSL AAWPVRATAS DPGWVHGRRE PVFVKPRNAF SDGDSALQFG | | |
| 01-07 | -Q-------- ------VA-- -----Y---- -------K-- ---------- | | |
| 02-11 | ---------- -----K---- --------C- -------K-- -----V--L- | | |
| 01-08 | ------D--- -----K---- -----R--C- ---L---K-- ---------- | | |
| | 886 ES6 | | |
| Lelystad | ELSESSSVIE FDRTKDAPVV DAPVDLTTSN EALSVVDPFE FAELKRPRFS | | |
| 01-07 | --------V- S--MN----- ---------- -------L-- ---------- | | |
| 02-11 | ---------- ---------- ---------- --F--C---- ---------- | | |
| 01-08 | ---------- --Q---TL-A ---------- ----A---S- -V--R---H- | | |
| | 936 | | |
| Lelystad | AQALIDRGGP LADVHAKIKN RVYEQCLQAC EPGSRATPAT REWLDKMWDR | | |
| 01-07 | ---------- ---------- Q---R----- ---------- --------E- | | |
| 02-11 | ---------- ---------- -------K-- ---------- K--------- | | |
| 01-08 | ---------- ---------- ---------- ---------- ---------- | | |
| | 986 | | |
| Lelystad | VDMKTWRCTS QFQAGRILAS LKFLPDMIQD TPPPVPRKNR ASDNAGLKQL | | |
| 01-07 | ---------- ---------- ---------- ---------- ---------- | | |
| 02-11 | ---------- ---------- ---------- ---------- ---------- | | |
| 01-08 | ---------- ---------- ---------- ---------- ---------- | | |
| | 1036 ES7 | | |
| Lelystad | VAQWDRKLSV TPPPKPVGPV LDQIVPPPTD IQQEDVTPSD GPPHAPDFPS | | |
| 01-07 | ---------M -S-Q----S- ---TAF--M- S---N----- -------C-- | | |
| 02-11 | -------F-- -----LA--- ---T-L---- A-R--A---- EL----HLL- | | |
| 01-08 | --R--K---- -----SA-L- ---T------ -----A---- -LS--S--S- | | |
| | 1086 | | |
| Lelystad | RVSTGGSWKG LMLSGTRLAG SISQRLMTWV FEVFSHLPAF MLTLFSPRGS | | |
| 01-07 | ---M--G--- -VF-S--F-- -*-------- ---V------ ---------- | | |
| 02-11 | ---M------ -IR------- -V--H----- ---------- ---------- | | |
| 01-08 | ----SW---- ---------- -AG------- ---Y------ I--------- | | |
| | 1136 | | |
| Lelystad | MAPGDWLFAG VVLLALLLCR SYPILGCLPL LGVFSGSLRR VRLGVFGSWM | | |
| 01-07 | ---------- ----S----H ----F----- ---------- ---------- | | |
| 02-11 | ---------- ----T----- T--------- ---------- ---------- | | |
| 01-08 | ---------- ---------- ---------- ---------- ---------- | | |
| | 1186 | | |
| Lelystad | AFAVFLFSTP SNPVGSSCDH DSPECHAELL ALEQRQLWEP VRGLVVGPSG | | |
| 01-07 | ---------- ---------- ---------- ---------- ---------- | | |
| 02-11 | --------V- ---------- ---------- T--------- ---------- | | |
| 01-08 | ---------- ---------- ---------- ---------- ---------- | | |
| | 1236 | | |
| Lelystad | LLCVILGKLL G | | |
| 01-07 | ---------- - | | |
| 02-11 | ---------- - | | |
| 01-08 | ---------- - | | |
| AMINO ACID ALIGNMENT OF TYPE 2 PRRSV NSP2 | | | |
| | 384 | | |
| 2332 nsp2 | AGKRARKARS CATATVAGRA LSVRETRQAK EHEVAGANKA EHLKHYSPPA EGNCGWHCIS AIANRMVNSK FETTLPERVR | SEQ ID NO: 5 | |
| NVSL-97 | ---------- GM-T---H-- -PA--IQ--- K--D---D-- V--R------ D--------- ---------- ---------- | SEQ ID NO: 6 | |
| PA8 | ---------- ---------- ---------- ---------- ---------- ---------- ---------- ---------- | SEQ ID NO: 7 | |
| HB1 | ---------- G--TM--H-- S-AH-----T K--G------ ----L----- ---------- --V------N ---------- | SEQ ID NO: 8 | |
| | 464 524 537 | | |
| 2332 nsp2 | PPDDWATDED LVNAIQILRL PAALDRNGAC TSAKYVLKLE GEHWTVTVTP GMSPSLLPLE CVQGCCGHKG GLGSPDAVEV | | |
| NVSL-97 | ---------- ---T----K- ---------- VG-------- ------S--L ---------- ------E--- ---P------ | | |
| PA8 | ---------- ---A------ ---------- ---------- ---------- ---------- ---------- ---------- | | |
| HB1 | ---------- ---T------ ---------- GG-------- ------S-N- ---------- ------E--- ---------- | | |
| | 544 | | |
| 2332 nsp2 | SGFDPACLDR LAEVMHLPSS AIPAALAEMS GDSDRSASPV TTVWTVSQFF ARHSGGNHPD QVRLGKIISL CQVIEDCCCS | | |
| NVSL-97 | F--------- ---------- V--------- --PNCP---- ---------- ---R-E---- ---------- ---V-E---H | | |
| PA8 | ---------- ---------- ---------- ---------- ---------- ---------- ---------- ---------- | | |
| HB1 | ---------- -LQ------- T-------L- D--N-PV--A AAT-----SY ---R----H- --C------- ---------H | | |
| | 624 | | |
| 2332 nsp2 | QNKTNRVTPE EVAAKIDLYL RGATNLEECL ARLEKARPPR VIDTSFDWDV VLPGVEAATQ TIKLPQVNQC RALVPVVTQK | | |
| NVSL-97 | ------A--- ----R--Q-- H---S----- I---RVC--S AA--F---N- -----G-S-- -T-QLH---- ---------E | | |
| PA8 | ---------- -----F---- ---------- ---------- ----F----- ---------- ---------- ---------- | | |
| HB1 | ------A--- -------Q-- ----S----- -K--RVS--G AA------N- --------H- -TEQLH--P- -T---P---E | | |
| | 704 | | |
| 2332 nsp2 | SLDNNSVPLT AFSLANYYYR AQGDEVRHRE RLTAVLSKLE KVVREEYGLM PTEPGPRPTL PRGLDELKDQ MEEDLLKLAN | | |
| NVSL-97 | P--KD----- ----S-C--P ---------- --NS------ G--------T --------A- -N--V----- --------V- | | |
| PA8 | ---------- ---------- ---------- ---------- ---------- --K------- ---------- ---------- | | |
| HB1 | P-GKD----- ----S-C--P ---N------ --NS------ E--L------ S-GL----V- -S-------- ---------- | | |
| | 784 | | |
| 2332 nsp2 | AQTTSDMMAW AVEQVDLKTW VKNYPRWTPP PPPPKVQPRK TKPVKSLPER KPVPAPRRKV GSDCGSPVSL GGDVPNSWED | | |
| NVSL-97 | --A--E---- -A------A- ---------- ----R----- --S-----GN ---------- R------ILM -DN--DGR-- | | |
| PA8 | ---------- -A-------- ---------- --S----L-- --------K- ---------- ---------- ---------- | | |
| HB1 | T-A--E---- -A------A- --S------- ----R----- --S------D ---------- R-G-----LM -DN---GS-- | | |
| | 864 | | |
| 2332 nsp2 | LAVSSPFDLP TPPEPATPSS ELVIVSSPQC IFRPATPLSE PAPIPAPRGT VSRPVTPLSE PIPVPAPRRK FQQVKRLSSA | | |
| NVSL-97 | -T-GG-L--S --S--M--L- -PALMPAL-Y -S--V-S--V L--V----R- ---------- --F-S---H- ----EEANL- | | |
| PA8 | ---------- ------I--- ---------- ---------- ---------- ---------- ---------- ---------- | | |
| HB1 | -T-GG-LNF- --S-PM-PM- -P-LTPAL-R VPKLM---DG S--V----R- ----M----- --FLS---H- ----EEANP- | | |
| | 944 | | |
| 2332 nsp2 | AAIPPYQDEP LDLSASSQTE YEASPPAPPQ SGGVLGVEGH EAEETLSEIS DMSGNIKPAS VSSSSSLSSV RITRPKYSAQ | | |
| NVSL-97 | -TTLTH---- ---------- -----LT-L- NM-I-E-G-Q ----V----- -TLND-N--P ---------- K-----H--- | | |
| PA8 | -A-----N-- ---------- ---------- ---------- ---------- ---------- ---------- ---------- | | |
| HB1 | TTTLTH-N-- ---------- -----L-SS- NMSI-EAG-Q ----V----- -ILNDTS--P ---------- K--------- | | |
| | 1024 | | |
| 2332 nsp2 | AIIDSGGPCS GHLQEVKETC LSVMREACDA TKLDDPATQE WLSRMWDRVD MLTWRNTSVY QAICTLDGRL KFLPKMILET | | |
| NVSL-97 | ---------- ---RRE--A- --I------- A--S------ ---------- --------A- --FRI----F E--------- | | |
| PA8 | ---------- ---------- ---------- ---------- ---------- ---------- -V------M- ---------- | | |
| HB1 | ---------- ----K-K-A- --I------- S--S------ ---------- --------A- -FRTLN---F E--------- | | |
| | 1104 | | |
| 2332 nsp2 | PPPYPCEFVM MPHTPAPSVG AESDLTIGSV ATEDVPRILE KIENVGEMAN QGPLAFSEDK PVDDQLVNDP RISSRRPDES | | |
| NVSL-97 | ---------- L--------- ---------- ---------G ----A---P- --L-TSFGEE --C--P-K-S WM---GF--- | | |
| PA8 | ---------- ---------- ---------- T--------- --G------- ---------- ---------- ---------- | | |
| HB1 | ---H--G--- L--------S ---------- ---------G --GDT--LL- ---S-PFKGG --C--PAKNS -M-P-ES--- | | |
| | 1184 | | |
| 2332 nsp2 | TSAPSAGTGG AGSFTDLPPS DGADADGGGP FRTVKRKAER LFDQLSRQVF DLVSHLPVFF SRLFYPGGGY SPGDWGFAAF | | |
| NVSL-97 | -T-------- -DLP------ --L---EW-- L---RK---- ---------- N--------- -H--KSDS-- ---------- | | |
| PA8 | ---------- ---------- ---------- ---------- ---------- ---------- ---------- ---------- | | |
| HB1 | II--P-D--- --------S- -SV--N---- L----T--G- -L----C--- S--------- -H--KSDS-- ---------- | | |
| | 1264 | | |
| 2332 nsp2 | TLLCLFLCYS YPAFGIAPLL GVFSGSSRRV RMGVFGCWLA FAVGLFKPVS DPVGAACEFD SPECRNILHS FELLKPWDPV | | |
| NVSL-97 | --F------- --F--FV--- ---------- ---------- ---------- ----T----- ------V--- ---------- | | |
| PA8 | ---------- ---------- ---------- ---------- ---------- ---------- ---------- ---------- | | |
| HB1 | --F------- --F--F---- ---------- ---------- ---------- ----T----- ------V--- ---------- | | |
| | 1344 | | |
| 2332 nsp2 | RSLVVGPVGL GLAILGRLLG | | |
| NVSL-97 | ---------- ---------- | | |
| PA8 | ---------- ---------- | | |
| HB1 | ---------- ---------- | | |
| NSP7 AMINO ACID ALIGNMENT WITH SELECTED NORTH AMERICAN TYPE 2 PRRSV ISOLATES | | | |
| Consensus #1 | SLTGALAMRLNDEDLDFLTKWTDFKCFVSASNMRNAAGQFIEAAYAKALRVELAQLVQVD | | |
| 11 | ..................M......................................... | 178 | SEQ ID NO: 9 |
| 12 | ..................M......................................... | 178 | SEQ ID NO: 10 |
| 13 | ..................M......................................... | 178 | SEQ ID NO: 11 |
| 14 | ..................M......................................... | 178 | SEQ ID NO: 12 |
| 15 | ............................................................ | 178 | SEQ ID NO: 13 |
| 16 | ..........T.......................................I......... | 178 | SEQ ID NO: 14 |
| 17 | ........K.S.........L...........................I........... | 178 | SEQ ID NO: 15 |
| 18 | ..................................................I......... | 178 | SEQ ID NO: 16 |
| 19 | ..................M......................................... | 178 | SEQ ID NO: 17 |
| 20 | ..........T.......................................I......... | 178 | SEQ ID NO: 18 |
| Consensus #1 | KVRGTLAKLEAFADTVAPQLSPGDIVVALGHTPVGSIFDLKVGSTKHTLQAIETRVLAGS | | |
| 11 | ............................................................ | 358 | |
| 12 | ............................................................ | 358 | |
| 13 | .............................R.............I................ | 358 | |
| 14 | ....V....................................................... | 358 | |
| 15 | ...........................................N................ | 358 | |
| 16 | .....M...................................................... | 358 | |
| 17 | ....V.............H........V.............I.NA............... | 358 | |
| 18 | ...........................................G......V......... | 358 | |
| 19 | ....V....................................................... | 358 | |
| 20 | .....M...................................................... | 358 | |
| Consensus #1 | KMTVARVVDPTPTPPPAPVPIPLPPKVLENGPNAWGDEDRLNKKKRRRMEALGIYVMGGK | | |
| 11 | ............................................................ | 538 | |
| 12 | ............................................................ | 538 | |
| 13 | ............................................................ | 538 | |
| 14 | ............................................................ | 538 | |
| 15 | .............L..............................R............... | 538 | |
| 16 | ............A...V..................................V..F..D.. | 538 | |
| 17 | R...........A...V...V...........S..............K...V........ | 538 | |
| 18 | .....................................G............TV..F..... | 538 | |
| 19 | ............................................................ | 538 | |
| 20 | ............A...V..................................V..F..D.. | 538 | |
| Consensus #1 | KYQKFWDKNSGDVFYEEVHNNTDEWECLRVGDPADFDPEKGTLCGHVTIENKAYHVYTSP | | |
| 11 | ............................................................ | 718 | |
| 12 | ............................................................ | 718 | |
| 13 | ............................................................ | 718 | |
| 14 | .........................................................I.. | 718 | |
| 15 | ..................................................DR........ | 718 | |
| 16 | ...................IS........T...V.....T.IQ...I...D.V.N.F... | 718 | |
| 17 | ...................D...A.....AD....L...R...........RP....A.. | 718 | |
| 18 | ...................D...A......................T...D.D.K..A.. | 718 | |
| 19 | .........................................................I.. | 718 | |
| 20 | ...................IS........T...V.....T.IQ...I...D.V.N.F... | 718 | |
| Consensus #1 | SGKKFLVPVNPENGRVQWE | | |
| 11 | ................... | 775 | |
| 12 | ................... | 775 | |
| 13 | ................... | 775 | |
| 14 | ................... | 775 | |
| 15 | ................... | 775 | |
| 16 | ..RR....A....R.A... | 775 | |
| 17 | ..R.....AD....KA... | 775 | |
| 18 | ..........S.S..A... | 775 | |
| 19 | ................... | 775 | |
| 20 | ..RR....A....R.A... | 775 | |
| NSP7 AMINO ACID ALIGNMENT WITH SELECTED TYPE1 PRRSV ISOLATES FROM THE U.S. | | | |
| Consensus #1 | TCCCTGACGGCTGCTCTAGCTTGCAAGTTGTCGCAGGCTGACCTTGATTTTTTGTCCAGC | | |
| 1 | .....A.........T................A........................... | 60 | SEQ ID NO: 19 |
| 3 | ..T..............G.......................T.....A...........T | 60 | SEQ ID NO: 20 |
| 4 | ....................C....................T.....C..C....T.... | 60 | SEQ ID NO: 21 |
| 5 | ...............................................C............ | 60 | SEQ ID NO: 22 |
| 6 | ...................................A........................ | 60 | SEQ ID NO: 23 |
| 7 | .....A...................................................... | 60 | SEQ ID NO: 24 |
| 8 | ..............C..............A.............................. | 60 | SEQ ID NO: 25 |
| 9 | .................G.................A...........C............ | 60 | SEQ ID NO: 26 |
| 10 | .........................................T.....C..C......... | 60 | SEQ ID NO: 27 |
| Consensus #1 | TTAACGAACTTCAAGTGCTTTGTATCTGCTTCAAACATGAAAAATGCTGCCGGCCAGTAC | | |
| 1 | ............................................................ | 120 | |
| 3 | .......................G...........T...........C............ | 120 | |
| 4 | ..G......C........G.............C.................T......... | 120 | |
| 5 | ...........................................................T | 120 | |
| 6 | ...............................................C..T......... | 120 | |
| 7 | ...............................................C............ | 120 | |
| 8 | ................................G.................T......... | 120 | |
| 9 | .............................C.................C............ | 120 | |
| 10 | ..G...............................................T......... | 120 | |
| Consensus #1 | ATTGAAGCAGCTTATGCCAAGGCCCTGCGCCAAGAGTTGGCCTCTCTAGTTCAGGTTGAC | | |
| 1 | ...........G..........................................A..... | 180 | |
| 3 | ........................T..................................T | 180 | |
| 4 | ............A....T.......................T.....G.....A...... | 180 | |
| 5 | ...........G................................C............... | 180 | |
| 6 | ...................G......A................................. | 180 | |
| 7 | ............................................................ | 180 | |
| 8 | ........................T................T..............C..T | 180 | |
| 9 | .....G...........T.G......A.....G........T.....G...........T | 180 | |
| 10 | .................T.......................T.....G.....A...... | 180 | |
| Consensus #1 | AAAATGAAAGGAATTTTGTCCAAGCTCGAGGCCTTTGCTGAAACAGCCACCCCGTCCCTT | | |
| 1 | ............G............................................... | 240 | |
| 3 | ............G............................................... | 240 | |
| 4 | ...T......................................................C. | 240 | |
| 5 | ....................T.....T.......................T......... | 240 | |
| 6 | ..G.........G............................................... | 240 | |
| 7 | ............G....A...........A....................T.....T... | 240 | |
| 8 | ..G.........................................C............... | 240 | |
| 9 | ...........G...C.A........T.................G.....T......... | 240 | |
| 10 | ............................................................ | 240 | |
| Consensus #1 | GACACAGGTGACGTGGTTGTTCTGCTTGGGCAACATCCTCACGGATCCATCCTCGATATT | | |
| 1 | ....T..........A............................................ | 300 | |
| 3 | .....................T..........G...........G..T............ | 300 | |
| 4 | .........................C.T................................ | 300 | |
| 5 | ...G........................................................ | 300 | |
| 6 | ............................................................ | 300 | |
| 7 | .....................T.............C........................ | 300 | |
| 8 | ..TC.....................................T..T............... | 300 | |
| 9 | ..............A........A................G................... | 300 | |
| 10 | ............................................................ | 300 | |
| Consensus #1 | AATGTGGGGACTGAAAGGAAAACTGTGTCCGTGCAAGAGACCCGGAGCCTAGGCGGCTCC | | |
| 1 | ............................................................ | 360 | |
| 3 | ..............................................A............. | 360 | |
| 4 | .....................................................T.TT... | 360 | |
| 5 | ................................................T....T..T... | 360 | |
| 6 | ..................................................T......... | 360 | |
| 7 | ........A.A................................................. | 360 | |
| 8 | .......................C.....T.............................. | 360 | |
| 9 | .....A.......................T..................T.......T..T | 360 | |
| 10 | .....................................................T..T... | 360 | |
| Consensus #1 | AAATTCAGTGTTTGCACTGTCGTGTCCAACACACCCGTGGACGCCTTAACCGGCATCCCA | | |
| 1 | ..............T................................G............ | 420 | |
| 3 | ............................................................ | 420 | |
| 4 | ..................................................T......... | 420 | |
| 5 | ..................................................T......... | 420 | |
| 6 | ....................T....................T.................. | 420 | |
| 7 | .......................T.................................... | 420 | |
| 8 | ..............T....................T........................ | 420 | |
| 9 | ..................................................T......... | 420 | |
| 10 | ..................................................T......... | 420 | |
| Consensus #1 | CTCCAGACACCAACCCCTCTTTTTGAGAATGGTCCGCGTCATCGCGGTGAGGAAGACGAT | | |
| 1 | .............................................A.C............ | 480 | |
| 3 | ....G....................................................... | 480 | |
| 4 | .......T...GG.A.............................T..C........T... | 480 | |
| 5 | .........................................C..T............... | 480 | |
| 6 | .............................................A.............. | 480 | |
| 7 | ......................................C......T.............. | 480 | |
| 8 | .............................................A.............C | 480 | |
| 9 | ................................C...........T..............C | 480 | |
| 10 | .......T...GG.A.............................T..C........T... | 480 | |
| Consensus #1 | CTTAAAGTCGAGAGGATGAAGAAACACTGTGTGTCCCTCGGCTTCCACAACATCAATGGC | | |
| 1 | ................................A........................... | 540 | |
| 3 | ..C.............................A........................... | 540 | |
| 4 | ........T.......A.G................T........................ | 540 | |
| 5 | ....G................................................T...... | 540 | |
| 6 | ............................................T............... | 540 | |
| 7 | ................AA..A...........T...........T.........C..... | 540 | |
| 8 | ..C.......................T.....A....................T.....A | 540 | |
| 9 | ....GT..........A....................................T...... | 540 | |
| 10 | ...................................T........................ | 540 | |
| Consensus #1 | AAAGTTTACTGCAAAATTTGGGACAAGTCTACCGGTGACACCTTTTACACCGATGATTCC | | |
| 1 | ..................................................G......... | 600 | |
| 3 | .....C.....T..G..C.....T.............................C...... | 600 | |
| 4 | ...........T.............................A...........C...... | 600 | |
| 5 | .............................C........T........T............ | 600 | |
| 6 | .....A...........C.....T.................................... | 600 | |
| 7 | ...........................................................T | 600 | |
| 8 | .G.........T...................................T............ | 600 | |
| 9 | .............................C.....A..T........T............ | 600 | |
| 10 | ...........T.............................A...........C...... | 600 | |
| Consensus #1 | CGGTACACCCAAGACCATGCTTTTCAGGACAGGTCAGCCGACTACAGAGACAGGGACTAT | | |
| 1 | ............................................................ | 660 | |
| 3 | .....T.....C...............................................C | 660 | |
| 4 | ..............T.......................T..................... | 660 | |
| 5 | ................T...A..C................................T... | 660 | |
| 6 | .....T.........T............................................ | 660 | |
| 7 | .............................T...........TC..........A.....C | 660 | |
| 8 | .....T........T..............T.............................. | 660 | |
| 9 | ................T...A..C................................T... | 660 | |
| 10 | ..............T.......................T..................... | 660 | |
| Consensus #1 | GAGGGTGTGCAAACCGCCCCCCAACAGGGCTTTGATCCAAAGTCTGAAACCCCTGTTGGC | | |
| 1 | ...............A.............A.............................. | 720 | |
| 3 | ..A.......................A................................. | 720 | |
| 4 | ......A...G..................A.............................. | 720 | |
| 5 | ......................................................A..... | 720 | |
| 6 | ..A......................................................... | 720 | |
| 7 | .....C.......................A.....C........C..G............ | 720 | |
| 8 | ..A.........G....T....C.A................................... | 720 | |
| 9 | ......................................................A..... | 720 | |
| 10 | ......A...G..................A.............................. | 720 | |
| Consensus #1 | ACTGTAGTGATCGGCGGTATTACGTATAACAGGTACCTGATCAAAGGTAAGGAGGTCCTG | | |
| 1 | .....T.............................T....................T... | 780 | |
| 3 | ....................C....................T.G................ | 780 | |
| 4 | .....T.............................T........................ | 780 | |
| 5 | .....G..............C....................................T.. | 780 | |
| 6 | .............................T........................A..... | 780 | |
| 7 | .................C.......................................... | 780 | |
| 8 | ......T...ATT.GC..G......................................... | 780 | |
| 9 | ..................G......................................T.. | 780 | |
| 10 | .....T.............................T........................ | 780 | |
| Consensus #1 | GTTCCCAAGCCTGACAACTGCCTTGAA | | |
| 1 | ..C........................ | 807 | |
| 3 | ..C....................C... | 807 | |
| 4 | ........................... | 807 | |
| 5 | ........................... | 807 | |
| 6 | ........................... | 807 | |
| 7 | ....GT.................C... | 807 | |
| 8 | ........A..C...C...... A..G | 807 | |
| 9 | ........................... | 807 | |
| 10 | ........................... | 807 | |

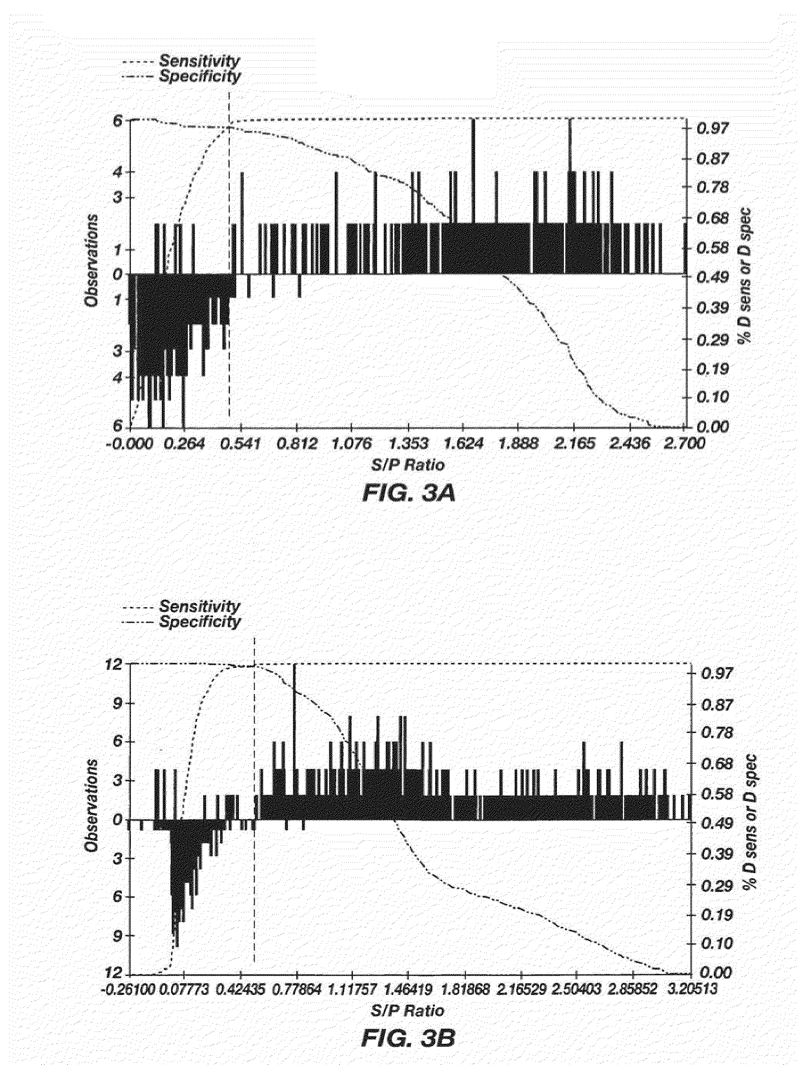

Further validation of the nsp7-based ELISA showed good sensitivity and specificity of the assay as determined by ROC analysis. The two-graph ROC plots of both Type I and Type II nsp7 ELISAs display the histograms of the uninfected and PRRSV-infected populations and demonstrates minimal overlap of the two populations (FIG. 3). The overlap between the two populations was attributed to eight samples from the Type I PRRSV infected population and nine samples from the Type II PRRSV infected population that had values below the established cutoff. Closer examination of these 17 samples revealed that all demonstrated strong background on the negative control well of ELISA plate, which suggests that the serum may contain other nonspecific components that interacted with the secondary antibody. In addition, eight of these samples were hemolyzed, which indicates that the serum collection and processing steps were not completed under optimal conditions. There are four samples from the negative population that demonstrated positive results on the Type I PRRSV ELISA and three samples from the negative population showing positive results on the Type II PRRSV ELISA. The IDEXX ELISA S/P values of these seven samples ranged from 0.2 to 0.3. This observation may support the practice by some veterinarians of using follow-up testing for any samples having an S/P value of greater than 0.20. We suspected that these samples might be from a herd that had a history of PRRSV infection, since the nsp7 ELISA was able to detect an antibody response to 202 dpi.

Serology is a standard diagnostic and surveillance method for determining if pigs have been exposed to PRRSV. Currently, the IDEXX PRRS ELISA is the most widely used serological assay for determining the serostatus of swine herds. However, positive IDEXX ELISA results in otherwise seronegative herds cause concern for producers, which necessitates the need for a variety of follow-up assays to verify that the result is either positive or negative. This indicates that there is still a need for a reliable assay to identify the serological status of single reactors compared to herd reactors. While there is no standard protocol to verify false positive serological results for PRRSV, most diagnostic laboratories use either the indirect fluorescent antibody (IFA) assay and/or virus neutralization assays. However, the results from both of these assays are affected by antigenic variation, and they may not detect a serological response against antigenically diverse PRRSV isolates, such as the European-like PRRSV strains, known as NA Type I isolates. The appearance of the Type I PRRSV isolates in the US also complicates the diagnosis of PRRSV as there is presently no standard serological assay that clearly differentiates between Type I and Type II strains of PRRSV. The movement of the swine industry toward strategies to eliminate or eradicate PRRSV will require an adequate serological diagnostic assay that can detect acute and persistently infected pigs, detect various strains of PRRSV and have the capacity to differentiate between Type I and Type II PRRSV isolates. Results generated in this study suggest that PRRSV nsp7 could be a potential new antigen for use in ELISA-based diagnostic assays. Especially, using a different target other than the N protein, any false positives specifically associated with the N antigen would be avoided.

In summary, our results showed that nsp1, nsp2 and nsp7 induced high levels of antibody response during the course of PRRSV infection. Among these three proteins, nsp7 is more suitable for diagnostic development with its characteristics of: 1) the nsp7 is expressed as soluble recombinant protein in bacterial culture, which is convenient for ELISA antigen preparation, especially when applied to diagnostic tests dealing with massive numbers of diagnostic samples; 2) the PRRSV nsp7 protein coding region is more homologous among different strains within the genotype in comparison to the other two immunogenic proteins, nsp1 and nsp2; 3) it is able to detect antibody responses later than 126 dpi. The nsp7-based ELISAs showed good sensitivity and specificity for identification and differentiation of Type I and Type II PRRSV. Furthermore, the nsp7 dual-ELISA resolved 98% samples with suspected false positive results of IDEXX ELISA. Therefore, nsp7-based ELISA may serve as an alternative or follow-up test of IDEXX ELISA.

### MATERIALS AND METHODS

Viruses and Cells: MARC-145 cells were cultured in Minimal Eagle's Medium (MEM; GIBCO BRL Life Technologies) with 10% fetal bovine serum (FBS) and antibiotics (100 units/ml penicillin, 20 ug/ml streptomycin). Cells were maintained at 37° C. in a humidified 5% CO₂ incubator. PRRSV strains SD01-08 (Type I) and VR2332 (Type II) were propagated on MARC-145 cells.

Antigen Production. Recombinant proteins were generated using SD01-08 (Type I) and VR-2332 (Type II) isolates. Based on the study of EAV, the PRRSV ORF1a encoded pp1a is predicted to be cleaved into eight products, nsp1 to nsp8. Nsp3 and nsp5 possess several predicted, nonimmunogenic hydrophobic domains (PepTool), so they were not considered further. The nsp6 is predicted to contain only 16 amino acids. A synthetic peptide made from these 16 amino acids was tested against sera from experimental infected pigs. When used in an ELISA format, there was no detectable antibody response. Therefore, only PRRSV nsp1, nsp2, nsp4, nsp7 and nsp8 were considered in this study. These nonstructural protein regions from VR2332 were expressed as recombinant proteins based on predicted cleavage sites in the pET-24 b vector (Novagen). Since nsp2 was expressed at low levels due to a C-terminal hydrophobic region, a C-terminal truncated portion was produced (13). Primers for amplifying each of the nonstructural proteins are listed in Table 1. The nsp7 encoding regions amplified from SD01-08 were cloned in the pET-28a (+) vector (Novagen). Recombinant proteins were expressed and purified and the purified fusion proteins were analyzed by SDS-PAGE and Western-blotting.

TABLE 1
Primers for PRRSV ELISA antigen expression

| | Nucleotide location | Predicted molecular | |
|---|---|---|---|
| | in the genome# | weight of the | |
| | (amino acid location | recombinant nsps | |
| Proteins* | in pp1a) | (KDa) | Primer sequences |
| NA nsp1 | 191-1339 | 42,950 | F: 5′ CGC [symbol] TCT GGG ATA CTT GAT CGG TG |
| | (1-383) | | SEQ ID NO: 28 |
| | | | R: 5′ CCG [symbol] GCC GTA CCA CTT GTG AC |
| | | | SEQ ID NO: 29 |
| NA nsp2P | 1340-3495 | 78,106 | F: 5′ CGC [symbol] GCT GGA AAG AGA GCA AG |
| | (384-1101) | | SEQ ID NO: 30 |
| | | | R: 5′ CCG [symbol] TCG AGT ATC ATT TTT GGG AGG AAC |
| | | | SEQ ID NO: 31 |
| NA nsp4 | 5618-6229 | 21,043 | F: 5′ CGC [symbol] GGT GCT TTC AGA ACT CGA AAG CC |
| | (1810-2013) | | SEQ ID NO: 32 |
| | | | R: 5′ CCG [symbol] TTC CAG TTC AGG TTT GGC AGC |
| | | | SEQ ID NO: 33 |
| NA nsp7 | 6788-7564 | 28,620 | F: 5′ CGC [symbol] TCT CTG ACT GGT GCC CTC GCT ATG |
| | (2200-2458) | | SEQ ID NO: 34 |
| | | | R: 5′ CCG [symbol] TTC CCA TTG AAC TCT TCC AT |
| | | | SEQ ID NO: 35 |
| EU nsp7 | 6417-7223 | 29,460 | F: 5′ G [symbol] TC CCT GAC GGC TGC TCT AGC TTG |
| | (2066-2334) | | SEQ ID NO: 36 |
| | | | R: 5′ GTG [symbol] TTT CAA GGC AGT TGT CAG GCT TGG |
| | | | SEQ ID NO: 37 |
| NA nsp8 | 7565-7696 | 4,872 | F: 5′ CGC [symbol] GCT GCA AAG CTT TCC GTG G |
| | (2459-2502) | | SEQ ID NO: 38 |
| | | | R: 5′ CCG [symbol] GTT TAA ACA CTG CTC CTT AGT C |
| | | | SEQ ID NO: 39 |

*NA = North American genotype (Type II); EU = European genotype (Type I); # numbers correspond to positions within the genome of Type II PRRSV, VR2332 (GenBank accession number U87392) or Type I PRRSV, SD01-08 (GenBank accession number DQ489311). Restriction enzyme sites are bold and Italicized.

Serum samples. For Type I PRRSV, a panel of serum samples (n=320) from 32 pigs experimentally inoculated with one of four different Type I PRRSV isolates, SD01-07, SD01-08, SD02-11 or SD03-15 (16) was used. They were collected at 7-day intervals for up to 85 days post inoculation (DPI). For Type II PRRSV, serial serum samples (n=1014) were obtained from 109 pigs experimentally infected with Type II PRRSV strain VR-2332. They were collected at 7-day intervals for the first two weeks and then at 14-day intervals for up to 202 days post inoculation (DPI). In addition, 1357 known PRRSV negative samples were obtained from negative control experimental pigs.

All of these serum samples, including 320 samples from Type I PRRSV infected animals, 1014 samples from Type II PRRSV infected animals, and 1357 samples from negative control animals were used for validation of the nsp7-based ELISA. Among these 1014 samples from Type II PRRSV infected animals, 510 serum samples were used for determining the kinetics of serological responses against pp1a proteins. To determine the ability of the nsp7-based ELISA to differentiate Type I and Type II PRRSV, a total of 470 known positive samples were tested with 215 samples from the Type I virus infected pigs and 255 samples from the Type II virus infected pigs.

In addition to samples of known status, the nsp7-based ELISA was evaluated using field samples, i.e., 1,107 serum samples collected from years 2007 to 2008 from 30 different farms in 10 different states (MN, CO, SD, WI, IL, WY, IA, KY, NE, and MO). These samples were also assayed in the IDEXX PRRS ELISA at the South Dakota Animal Disease Research and Diagnostic Laboratory (SD ADRDL). In addition, 100 IDEXX ELISA suspected false positive samples were also obtained from the SD ADRDL and tested in the nsp7-based ELISA.

PRRSV nsp antigen-based ELISA. The nsp antigen-based ELISA was performed using Immulon 2 HB 96-well microtiter plates (Thermo Labsystems, Franklin, Mass.). A single lot of internal quality control serum samples, generated from experimentally infected pigs, was used to establish the standards of: high positive (optical density ˜1.9-2.1), low positive (optical density ˜0.6-0.7) and negative (optical density<0.2). The optimal dilution of the recombinant protein was experimentally determined so that the control serum sample generated an optical density (OD) as the established standard. The recombinant protein was diluted in 15 mM sodium carbonate-35 mM sodium bicarbonate (ACB), pH 8.8. The plates were coated with 100 μl (˜2 ug/ml) of the diluted protein in columns 1, 3, 5, 7, 9, and 11. Columns 2, 4, 6, 8, 10, and 12 were coated with 100 μl of ACB as a background control. For the nsp7-based ELISA, columns 1, 4, 7, 10 were coated with Type I PRRSV nsp7 antigen (SEQ ID NO:43), columns 2, 5, 8, 11 were coated with Type II PRRSV nsp7 antigen, and columns 3, 6, 9, 12 were coated with ACB as a background control. The plates were incubated for 1 h at 37° C. and then blocked with 10% w/v powdered dry milk in PBS containing 0.05% Tween 20 (PBST) at 4° C. overnight. The following day, plates were washed with 300 μl of PBST. Test and control sera were diluted 1:50 with PBST containing 5% milk in PBST, and 100 μl of the dilution were added into the well. The plates were incubated for 1 h at 37° C. Plates were then washed, and 100 ul of goat anti-swine horseradish peroxidase conjugate (KPL, Gaithersburg, Mass.) was added to all wells. Plates were incubated for 1 h at 37° C., washed, and then 100 μl of ABTS peroxidase substrate (KPL, Gaithersburg, Md.) was added to all wells. Color development was observed until the positive control reached a standard OD and then stopped by the addition of 100 μl of ABTS stop solution (KPL, Gaithersburg, Mass.). Color development was quantified by reading at 405 nm with an EL800 microplate reader (BioTek Instruments Inc., Winooski, Vt.) controlled by XChek Software (IDEXX Laboratories). The raw plate data were copied to an Excel spreadsheet to calculate the sample to positive (S/P) ratios using the following formula: S/P=(OD of sample−OD of buffer)/(OD of positive control−OD of buffer). Statistical analysis was performed using GraphPad InStat version 3.06 (GraphPad Software, San Diego, Calif.). Correlation of determination between mean S/P ratios was analyzed using Pearson R correlation analysis assuming Gaussian distribution of data.

Validation of nsp7-based ELISA: (i) Cutoff determination, diagnostic sensitivity, and diagnostic specificity. To accurately assess the diagnostic sensitivity and diagnostic specificity of the nsp7 ELISA, 2,691 serum samples from individual animals with established PRRSV status were analyzed using the nsp7 dual-ELISA and the IDEXX ELISA. The negative-testing (non-infected) validation population was composed of samples from individual animals of negative control groups. The positive-testing (infected) validation population was composed of samples from experimentally infected animals (refer to previous “serum samples” section). Receiver Operating Characteristic (ROC) analysis methodology assessment was performed using GRAPH ROC software (14) (Version 2.0). (ii) Measurement of repeatability. The repeatability of the nsp7 dual-ELISA was assessed by running the same lot of internal quality control sera. Within-plate precision was calculated from 40 replicates on one plate, within-run precision was calculated using one serum on 10 plates in one run, and between-run precision was calculated from at least one serum in 10 different runs. Means, standard deviations (sd), percent coefficient of variation (% CV) values, and Levey-Jennings control charts were calculated using Control Chart Pro Plus software (version 7.12.24; ChemSW). (iii) Calculation of reactivity ratio (r). For each positive sample, an r value, representing the log 10 of the ratio obtained by dividing the S/P ratio observed in the Type I nsp7 ELISA by the S/P ratio observed in the Type II nsp7 ELISA, was calculated. Thus, r values of >0 represent positives in the Type I nsp7 ELISA, and r values of <0 represent positives in the Type II nsp7 ELISA.

Immunofluorescence Assay (IFA). MARC-145 cells were grown in cultures for 3 to 4 days to confluence on 96-well cell culture plates (BD Biosciences, San Jose, Calif.). Every other column was infected with PRRSV (5×10³ 50% tissue culture infective doses/ml), and the plates were incubated for an additional 18 to 24 h. The plates were then fixed with 300 μl of 50% (vol/vol) acetone/methanol per well for 20 min at −20° C., air dried and frozen with a desiccant at −20° C. until use. Serum samples to be assayed were diluted 1:20 and 1:40 with PBS, and 100 μl of each dilution was transferred to paired wells of PRRSV-infected and uninfected MARC-145 cells. The plates were incubated at 37° C. for 1 hour and then washed three times with 300 μl of PBS. Then, 30 μl of fluorescein isothiocyanate (FITC)-labeled goat anti-swine immunoglobulin G (41.7 ug/ml; KPL) was added to each well. The plates were incubated at 37° C. for 1 hour, and washed with PBS three times. The cells were examined for specific fluorescence with an inverted microscope and a UV light source (Nikon Eclipse TS100).

### Results

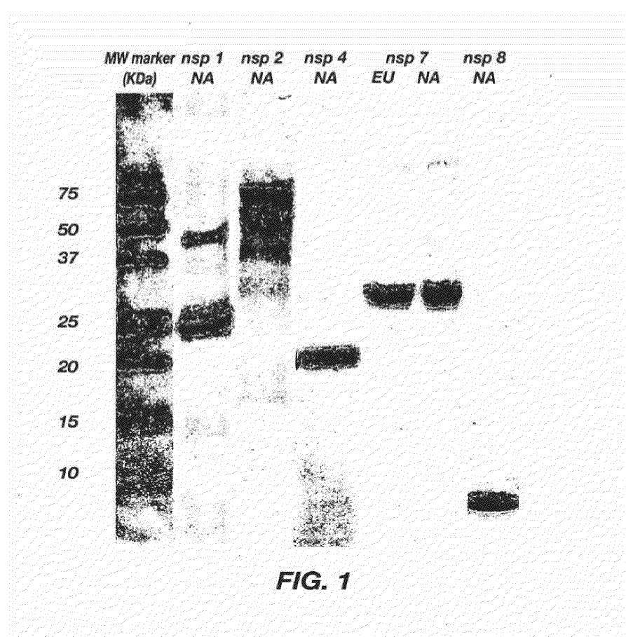

Antigen production. DNA fragments corresponding to all or portions of nsp1, nsp2, nsp4, nsp7, nsp8 from Type II PRRSV VR2332, and nsp7 from Type I PRRSV SD01-08 were cloned and expressed in E. coli. Nsp4, nsp7 and nsp8 were expressed at high levels and could be purified in soluble forms. In contrast, recombinant nsp1 and nsp2 formed inclusion bodies and a protein refolding step was performed. The purity of the recombinant proteins was evaluated using sodium dodecyl sulfate-polyacrylamide gel electrophoresis followed by Coomassie blue staining. As shown in FIG. 1, all of the His-tagged recombinant proteins migrated according to their predicted sizes listed in Table 1. The identity of each protein was confirmed by Western blot analysis with monoclonal anti-His antibody and the specific swine anti-sera (data not shown).

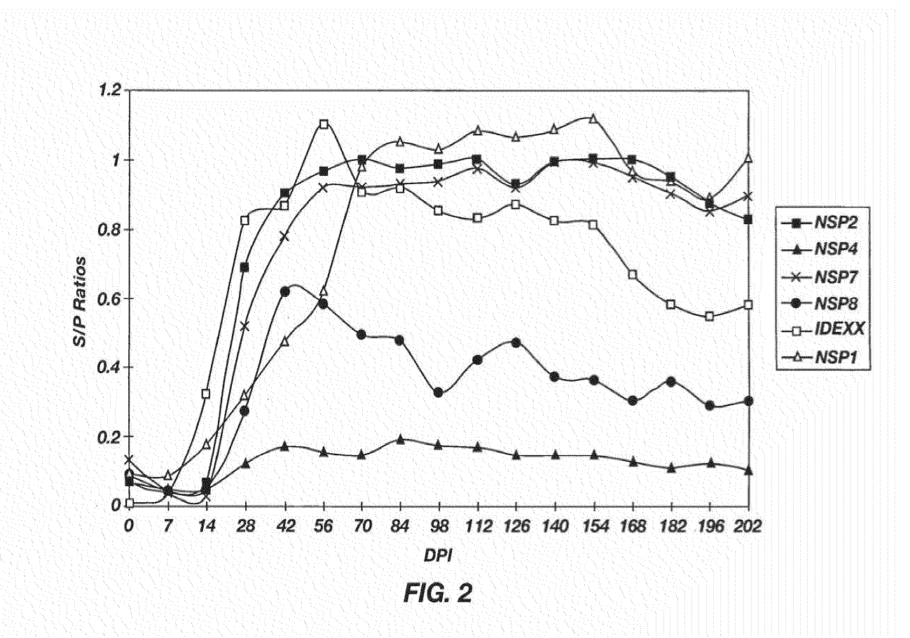

Determine the kinetics of serological responses against pp1a proteins. Testing of the serological response to nsp1, 2, 4, 7 and 8 were conducted using 510 serum samples that were collected from 30 pigs experimentally infected with Type II isolate VR2332. As shown in FIG. 2, nsp4 reacted weakly with the swine immune sera. Nsp8 had a lower antibody response in comparison to nsp1, 2 and 7, and the titer dropped after 98 dpi. Nsp1, nsp2 and nsp7 reacted strongly with pig immune sera. The antibodies specific to these proteins were detected as early as 14 dpi, and the responses lasted more than 202 dpi. Interestingly, there was a decrease of antibody titer over time to N protein after 126 dpi, while the antibody titers of nsp1, nsp2 and nsp7 remained relatively high. We further performed detailed comparison of these three antigens including their sensitivity to early seroconversion and correlation with N protein, which is the antigen used in the IDEXX HerdChek® PRRS 2XR ELISA. At 7 dpi, all samples tested negative (S/P<0.2) by all antigens. At 14 dpi, approximately 65% of the samples were detected as seropositive (S/P>0.5) by all ELISAs; at 21 dpi, all samples were identified as positive (S/P>0.5) irrespective of the antigen used for detection (Table 2). The correlation between the PRRSV nsp ELISA and IDEXX ELISA was evaluated using Pearson R correlation analysis. During the first 126 days post-infection, the ELISA results from nsp2 and nsp7 correlated well with those of the IDEXX ELISA (R=0.91 between nsp2 and IDEXX; R=0.84 between nsp7 and IDEXX). In contrast, statistical correlation of nsp1 ELISA to IDEXX ELISA was 0.57, which is lower than that of nsp2 and nsp7.

TABLE 2
Comparison of seroconversion detected by
PRRSV nsp ELISA and IDEXX ELISA

| | number of seropositive^{a} animals detected/ | | |
|---|---|---|---|
| | total number of experimental animals | | |
| Method | 7 dpi^{b} | 14 dpi | 21 dpi |
| nsp1 ELISA | 0/30 | 22/30 | 30/30 |
| nsp2 ELISA | 0/30 | 21/30 | 30/30 |
| nsp7 ELISA | 0/30 | 23/30 | 30/30 |
| IDEXX ELISA | 0/30 | 23/30 | 30/30 |

^{a}S/P > 0.5 is considered as seropositive for nsp ELISAs, while S/P > 0.4 is considered as seropositive for IDEXX ELISA.
^{b}dpi: days post infection.

Cutoff determination, diagnostic sensitivity, and diagnostic specificity of nsp7-based ELISA. An nsp7-based ELISA was chosen to further evaluate as a serology diagnostic assay for detection and differentiation of Type I and Type II PRRSV. The robustness and repeatability of the nsp7-based ELISA were assessed to determine its potential for diagnostic application. Recombinant nsp7 antigens were prepared from the Type I virus, SD01-08, and the Type II virus, VR2332. Serum samples from a known positive population (Type I and Type II PRRSV-infected) of 1,334 animals and 1,357 serum samples from a known negative population (PRRSV-uninfected) were analyzed with the nsp7-based ELISA and the IDEXX ELISA. GRAPH ROC software was used for ROC analysis of nsp7-based ELISAs to determine an optimized cutoff that maximizes both the diagnostic specificity and diagnostic sensitivity of these assays. An optimized cutoff that maximized the efficiency of the assay was calculated at an S/P of 0.51 for the Type I nsp7 ELISA, and S/P of 0.52 for the Type II nsp7 ELISA (FIG. 3). A diagnostic sensitivity of 97.4% (95% confidence interval of 94.4% to 99.1%) and a corresponding diagnostic specificity of 98.3% (95% confidence interval of 94.1% to 98.7%) were calculated for the Type I nsp7-ELISA, while a diagnostic sensitivity of 99.8% (95% confidence interval of 99.4% to 100%) and a corresponding diagnostic specificity of 99.3% (95% confidence interval of 97.1% to 99.5%) were calculated for the Type II nsp7-ELISA. When the S/P of 0.4 cutoff determined by IDEXX was used, the diagnostic sensitivity was 97.4% (95% confidence interval of 86% to 99.9%) and the diagnostic specificity of the IDEXX ELISA was 99.6% (95% confidence interval of 97.8% to 99.9%). These results indicate that the nsp7-based ELISA is very comparable to the IDEXX ELISA.

Repeatability of the nsp7-based ELISA. The precision of the IDEXX ELISA and the nsp7-based ELISA were compared using internal-control sera. The percent coefficient of variation (% CV) was calculated using the protocol described earlier (9). The IDEXX ELISA within-plate % CV was 7.1, the % CV between plates in one run was 11.9, and the % CV between runs was 14.8. The nsp7-based ELISA appears to have similar variability to the IDEXX ELISA. The Type I nsp7 ELISA within-plate % CV was 6.5, the % CV between plates in one run was 11.9, and the % CV between runs was 17.1, while the Type II nsp7 ELISA within-plate % CV was 2.3, the % CV between plates in one run was 5.4, and the % CV between runs was 9.5. These results suggest that the nsp7-based ELISA is highly repeatable in diagnostic applications.

TABLE 3
Comparison of assay repeatability between IDEXX and nsp7 dual-ELISA

| | Repeatability results (% CV)* | | |
|---|---|---|---|
| Assay | Within plate | Within run | Between runs |
| IDEXX ELISA | 7.1 | 11.9 | 14.8 |
| Type I nsp7 ELISA | 6.5 | 11.9 | 17.1 |
| Type II nsp7 ELISA | 2.3 | 5.4 | 9.5 |

*Values listed are % CV of high-level positive internal control serum

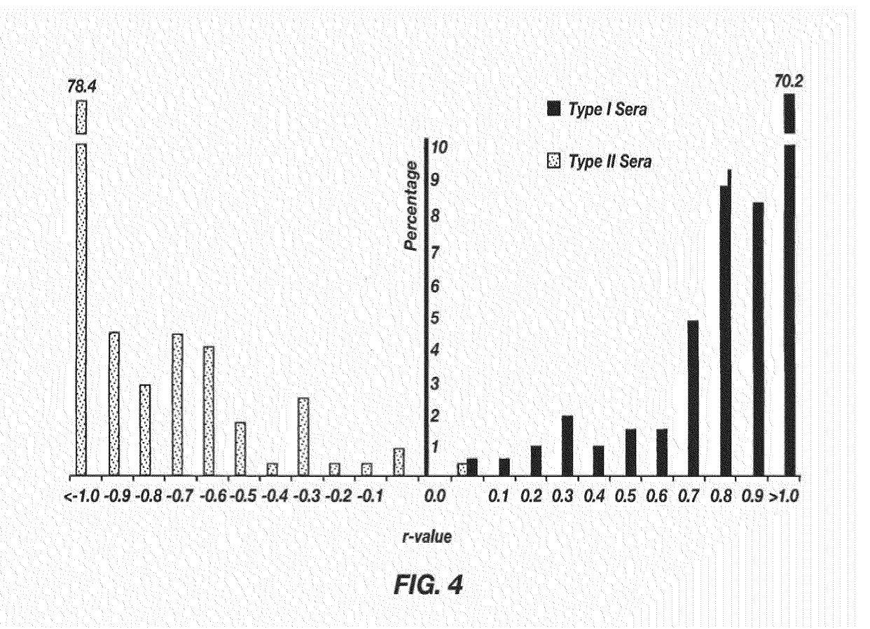

Application of nsp7-based ELISA for the differentiation of Type I and Type II PRRSV. To determine if the nsp7-based ELISA can be used to differentiate the serum antibodies produced in response to infection with Type I and Type II viruses, a total of 470 known positive samples were tested. The results showed that all of the samples from Type I virus-infected pigs tested positive using the Type I nsp7 ELISA (S/P>0.51), and no specific antibody responses were detected in sera samples from Type I PRRSV infected animals in the Type II nsp7 ELISA (S/P<0.52; see FIG. 4). Similarly, 254 out of 255 samples from Type II virus-infected pigs tested positive using the Type II nsp7 ELISA (S/P>0.52), and only one sample from Type II PRRSV infected animals tested positive in the Type I nsp7 ELISA (S/P>0.51; see FIG. 4). These results indicate that the nsp7-based ELISAs are specific for identifying the antibody response within the genotype and are capable of differentiating antibody responses to Type I and Type II PRRSV infection.

TABLE 4
Comparison of sensitivity and specificity of the
nsp7 dual-ELISA and IDEXX ELISA for the detection
of antibodies against Type I and Type II PRRSV

| | IDEXX | Type I nsp7 | Type II nsp7 |
|---|---|---|---|
| Characteristics | ELISA | ELISA | ELISA |
| Optimized cut off (S/P) | 0.4 | 0.51 | 0.52 |
| Diagnostic sensitivity (%) | 97.4 | 97.4 | 99.8 |
| 95% confidence interval | 86-99.9 | 94.4-99.1 | 99.4-100 |
| Diagnostic specificity (%) | 99.6 | 98.3 | 99.3 |
| 95% confidence interval | 97.8-99.9 | 94.1-98.7 | 97.1-99.5 |

Comparison of the nsp7-based ELISA with the IDEXX ELISA for the detection of pigs infected with field viruses. We used a broad spectrum of field serum samples (1,107 samples) submitted to the SD ADRDL to determine if the nsp7-based ELISAs could be applicable for detecting serum antibody response from pigs infected with various genetically different field strains. Since the source of field sera samples was unknown (whether pigs were infected by Type I or Type II PRRSV), we used antigens from both Type I and Type II PRRSV and designated this test as the nsp7 dual-ELISA. When comparing the nsp7 dual-ELISA with the IDEXX ELISA, 490 out of 502 (97.6%) IDEXX positive samples were tested as positive by the nsp7 dual-ELISA, and 590 out of 605 (97.5%) IDEXX negative samples were tested as negative by the nsp7 dual-ELISA. We further investigated the application of the nsp7-based ELISA in samples with unexpected positive IDEXX results. An unexpected positive result was defined as IDEXX positive but negative when tested by IFA and no evidence of exposure to PRRSV. One hundred samples with suspected false positive IDEXX ELISA results were obtained from the SD ADRDL and these samples were verified by IFA as seronegative. The nsp7 dual-ELISA results showed that 98 samples (98%) tested as negative (Table 5).

TABLE 5
Evaluation of field sera and samples with IDEXX ELISA
unexpected positive results using the nsp7 dual-ELISA

| | | Total | | | Nsp7 |
|---|---|---|---|---|---|
| | | number | | | dual- |
| Serum | | of | IDEXX | IFA | ELISA |
| group | Results | samples | positive | positive | positive |
| 1 | IDEXX positive; | 502 | 502 | NA* | 490 |
| | 97.6% tested | | | | |
| | positive in nsp7 | | | | |
| | dual-ELISA | | | | |
| 2 | IDEXX negative; | 605 | 0 | NA | 15 |
| | 97.5% tested | | | | |
| | negative in nsp7 | | | | |
| | dual-ELISA | | | | |
| 3 | Unexpected IDEXX | 100 | 100 | 0 | 2 |
| | ELISA positive | | | | |
| | result; 98% showing | | | | |
| | negative in nsp7 | | | | |
| | dual-ELISA | | | | |

*NA: not applicable. IFA has not performed for these specific samples.

### DISCUSSION

The current study aimed to determine the humoral immune response to the PRRSV nonstructural proteins and to develop new tools for identification of PRRSV infected animals. Previous studies of the humoral immune response to PRRSV have focused mainly on detection of antibodies to viral structural proteins, especially nucleocapsid. Several studies showed that certain nonstructural proteins, such as nsp1 and nsp2 are highly immunogenic. Antibody responses to linear epitopes in nsp2 have been reported to appear within 1-4 weeks of infection in Type I and Type II PRRSV strains. Johnson et al. observed robust and rapid cross-reactive antibody responses induced by nsp1 and nsp2 to vaccine and field isolates, and substantially higher levels of immunoreactivity related to conformational epitopes. In this study, our data demonstrated that nsp7 is also highly immunogenic. Analysis of the kinetics of antibody response showed that response to nsp7 is comparable to antibody response to nsp1 and nsp2 as well as antigens used in the commercial IDEXX ELISA. As indicated by Johnson et al., nsps are available from the earliest time of infection for presentation to the immune system in the context of major histocompatibility complex (MHC) class I antigen-presentation pathways. As cytolytic infection also releases viral proteins into interstitial spaces, it is hypothesized that a pronounced antibody response, equivalent to the immune response to structural proteins, would be generated to nonstructural proteins. One intriguing feature of the antibody response to nsp antigens was the sustained antibody titers over a 202 day period of infection, while the antibody response to IDEXX antigen, N protein, showed a gradual decay in titers after 126 dpi. The mechanism for sustained levels of nsp antigen may reflect the long-term retention and presentation of nsp to the immune system.

To select an antigen for diagnostic test development, we compared the correlation between the PRRSV nsp ELISA with IDEXX ELISA. Our results showed that nsp2 and nsp7-based ELISA had higher correlation with those of the IDEXX ELISA. We further compared the amino acid sequences of nsp2 and nsp7. Our previous studies showed that the PRRSV nsp2 region is highly variable within and between genotypes with 70.6%-91.6% amino acid identity within Type I PRRSV and 74.9%-95.6% amino acid identity within Type II PRRSV, but only 33.8% identity between Type I and Type II genotypes. The central region of the nsp2 contains hypervariable domains with insertions and deletions, and most identified B-cell epitopes are located in these regions. In contrast, the nsp7 is relatively conserved within each genotype and is divergent between genotypes Amino acid sequence comparisons showed that nsp7 shares 96.7%-97.4% amino acid identity within Type I PRRSV and 84.9%-100% amino acid identity within Type II PRRSV, but only about 45% identity between Type I and Type II genotypes. These results suggest that the nsp7-based ELISA could be able to detect genotype specific anti-nsp7 antibody responses.

Further validation of the nsp7-based ELISA showed good sensitivity and specificity of the assay as determined by ROC analysis. The two-graph ROC plots of both Type I and Type II nsp7 ELISAs display the histograms of the uninfected and PRRSV-infected populations and demonstrates minimal overlap of the two populations (FIG. 3). The overlap between the two populations was attributed to eight samples from the Type I PRRSV infected population and nine samples from the Type II PRRSV infected population that had values below the established cutoff. Closer examination of these 17 samples revealed that all demonstrated strong background on the negative control well of ELISA plate, which suggests that the serum may contain other nonspecific components that interacted with the secondary antibody. In addition, eight of these samples were hemolyzed, which indicates that the serum collection and processing steps were not completed under optimal conditions. There are four samples from the negative population that demonstrated positive results on the Type I PRRSV ELISA and three samples from the negative population showing positive results on the Type II PRRSV ELISA. The IDEXX ELISA S/P values of these seven samples ranged from 0.2 to 0.3. This observation may support the practice by some veterinarians of using follow-up testing for any samples having an S/P value of greater than 0.20. We suspected that these samples might be from a herd that had a history of PRRSV infection, since the nsp7 ELISA was able to detect an antibody response to 202 dpi.

Serology is a standard diagnostic and surveillance method for determining if pigs have been exposed to PRRSV. Currently, the IDEXX PRRS ELISA is the most widely used serological assay for determining the serostatus of swine herds. However, positive IDEXX ELISA results in otherwise seronegative herds cause concern for producers, which necessitates the need for a variety of follow-up assays to verify that the result is either positive or negative. This indicates that there is still a need for a reliable assay to identify the serological status of single reactors compared to herd reactors. While there is no standard protocol to verify false positive serological results for PRRSV, most diagnostic laboratories use either the indirect fluorescent antibody (IFA) assay and/or virus neutralization assays. However, the results from both of these assays are affected by antigenic variation, and they may not detect a serological response against antigenically diverse PRRSV isolates, such as the European-like PRRSV strains, known as NA Type I isolates. The appearance of the Type I PRRSV isolates in the US also complicates the diagnosis of PRRSV as there is presently no standard serological assay that clearly differentiates between Type I and Type II strains of PRRSV (7, 28). The movement of the swine industry toward strategies to eliminate or eradicate PRRSV will require an adequate serological diagnostic assay that can detect acute and persistently infected pigs, detect various strains of PRRSV and have the capacity to differentiate between Type I and Type II PRRSV isolates. Results generated in this study suggest that PRRSV nsp7 could be a potential new antigen for use in ELISA-based diagnostic assays. Especially, using a different target other than the N protein, any false positives specifically associated with the N antigen would be avoided.

In summary, our results showed that nsp1, nsp2 and nsp7 induced high levels of antibody response during the course of PRRSV infection. Among these three proteins, nsp7 is more suitable for diagnostic development with its characteristics of: 1) the nsp7 is expressed as soluble recombinant protein in bacterial culture, which is convenient for ELISA antigen preparation, especially when applied to diagnostic tests dealing with massive numbers of diagnostic samples; 2) the PRRSV nsp7 protein coding region is more homologous among different strains within the genotype in comparison to the other two immunogenic proteins, nsp1 and nsp2; 3) it is able to detect antibody responses later than 126 dpi. The nsp7-based ELISAs showed good sensitivity and specificity for identification and differentiation of Type I and Type II PRRSV. Furthermore, the nsp7 dual-ELISA resolved 98% samples with suspected false positive results of IDEXX ELISA.

From these properties, it is clear that the nsp7-based ELISA of the disclosure is convenient with respect to antigen production, and it is reliable, economical, and highly sensitive and specific. Thus, it is considered to be a useful tool for routine diagnostics, epidemiological surveys, and outbreak investigations.

One aspect of the invention is the application of a PRRSV non-structural protein in a serological assay and the ability to differentiate antibody responses against the two different genotypes of PRRSV. The cause of the unexpected positive, or “false positive”, serological results obtained when using the IDEXX PRRS ELISA is believed to be at least partially due to the presence of an epitope on the nucleocapsid protein of PRRSV that is not totally unique to PRRSV. The use of an alternative target antigen, such as nsp7, is believed to be a reasonable solution to prevent, or at least avoid, the false positive problem. Alternatively, the IDEXX PRRS ELISA may be used in combination with the nsp7-based ELISA.

The disclosed assay improves the diagnosis of a very important disease of swine, and also has substantial value for use in epidemiological studies.

Previous studies of the humoral immune response to PRRSV have mainly focused on detection of antibodies to structural proteins (Oleksiewicz et al., 2002; Loemba et al., 1996; Murtaugh et al., 2002; Meulenberg 1995), especially on the nucleocapsid protein. The PRRSV non-structural proteins play a critical role in virus replication and recent studies have indicated that some, but not all, nsps are highly immunogenic. Antibody responses to linear epitopes in nsp2 have been reported to appear within 1-4 weeks of infection in Type 1 and Type 2 PRRSV strains (de Lima et al., 2006; Oleksiewicz et al., 2001a, b, 2002). Johnson et al. (2007) observed a robust and rapid cross-reactive antibody response induced by nsp1 and nsp2 to vaccine and field isolates, and substantially higher levels of immunoreactivity related to conformational epitopes. The present invention demonstrates that besides nsp1 and nsp2, nsp7 also induces high levels of antibody response.

As indicated by Johnson et al. (2007), nonstructural proteins are available from the earliest time of infection for presentation to the immune system in the context of major histocompatibility complex (MHC) class I antigen-presentation pathways. As cytolytic infection also releases viral proteins into interstitial spaces, it is hypothesized that a pronounced antibody response, equivalent to the immune response to structural proteins, would be generated to nonstructural proteins. In comparison to the N protein, differences in the kinetics of the immune responses to N and nsp1, nsp2 and nsp7 proteins were observed at the later stage of infection (after 126 dpi). Antibody titers to nsp1, nsp2 and nsp7 remained at similar levels at the early stages of infection, but there was a substantial decrease of antibody levels for the N protein at later stages of infection. This result indicates that nsp7 is a better detector for PRRSV persistence.

The present results demonstrate that nsp7 is surprisingly well-suited for use in a diagnostic test or kit due to the fact that:

1) nsp7 is expressed as a soluble recombinant protein in bacterial culture, which is convenient for ELISA antigen preparation, especially when applied to the diagnostic tests dealing with massive numbers of diagnostic samples;
2) the PRRSV nsp7 protein coding region is more homologous among different strains within the genotype; and
3) an ELISA using nsp7 detects an earlier antibody response, which correlates well with IDEXX ELISA results.

Thus, a kit in accordance with the invention disclosed herein may include a solid support (e.g., a microtiter plate) for immobilizing capture reagents. The kit may also include, in a preferred embodiment, a detection means (e.g., colormetric or fluorimetric means, or other suitable means) for detecting antibodies. The kit may further include instructions, and may include standards against which samples may be measured.

The kits of the invention may be sold through establishments selling or providing diagnostic kits. Methods of providing diagnostic services may also be implemented where samples from animals suspected of being infected are sent to a diagnostic testing lab, and the methods of the invention are used diagnostically and to determine the type or types of infection.

Monitoring the serostatus of PRRSV-negative or low-prevalence herds is important to the swine industry. When the IDEXX ELISA is used as a screening tool, unexpected positive results from samples in negative-testing herds may require additional tests to resolve the problem. The nsp7 based Dual-ELISA described herein shows good sensitivity and specificity for the identification and/or differentiation of Type 1 and Type 2 PRRSV clinical samples. Therefore, a nsp7-based ELISA provides an alternative test to the IDEXX ELISA.

The nsp 1, nsp2 and nsp7 induced higher antibody responses than the other nsps and can be detected as early as 14 dpi, while lasting more than 202 dpi. Antibodies to nsp8 can be detected at 21 dpi, but the titer remains low (FIG. 2). The nsp7-based ELISA performed better than the other nsps-based ELISAs in regard to antigen preparation and diagnostic test application (Table IV).

Using nsp7 recombinant protein as the antigen, a dual enzyme-linked immunosorbent assay (nsp7 Dual-ELISA) for the simultaneous detection and differentiation of serum antibodies directed against Type 1 and Type 2 PRRSV is provided herein. Alternatively, a single nsp7 antigen ELISA may be used or a mixture of nsp7 antigens derived from both type I and type II may be used simultaneously, depending on the goals that are to be achieved.

Taken together, the data of the present disclosure indicate that the PRRS nsp7 Dual-ELISA described herein is the first differential ELISA for PRRSV serology based on non-structural proteins. It is convenient with respect to antigen production, and it is reliable, economical, and highly sensitive and specific. Thus, it is considered to be a potential tool for routine diagnostics, epidemiological surveys, and outbreak investigations.

While the invention is illustrated using full length or nearly full length nsp7 protein, in light of the present disclosure it will be now recognized that the assay may also utilize fragments or epitopes of nsp7, which may be produced using methods known in the art. For example, an epitope region or fragment of nsp7 may be constructed and expressed using any expression vector, such as pET-28a (+) (Novagen). Likewise, one or more flexible peptide linkers (e.g., GGGGS) may be added between the epitopes or fragments to help display the epitopes or fragments. Such epitopes or fragments may be prepared using forward and reverse primers designed using methods known in the art. The primers may optionally contain one or more restriction sites to facilitate cloning of the epitope or fragment into the expression vector. The recombinant proteins, epitopes or fragments may be expressed in any suitable expression system, including, but not limited to, E. coli BL21 cells, mammalian cell lines (e.g., Chinese hamster ovary cells, HEK293 cells, or HELA cells), insect cells (e.g. using baculovirus expression vectors), yeast (e.g., Pichia pastoris,) or any other system, to produce a recombinant nsp7 protein, epitope or fragment. Optionally the nsp7 protein, epitope or fragment may contain other features, such as a histidine tag that facilitates purification by nickel-affinity chromatography.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including Genbank accession numbers, publications, patents, and patent applications, cited herein are hereby incorporated by reference to the same extent as if each reference were individually and specifically indicated to be incorporated by reference and were set forth in its entirety herein, including the following:
1. Allende, R., T. L. Lewis, Z. Lu, G. F. Rock, A. Kutish, A. Ali, A. R. Doster, and F. A. Osorio. 1999. North American and European porcine reproductive and respiratory syndrome viruses differ in non-structural protein coding regions. J. Gen. Virol. 80:307-315.
2. Bautista, E. M., S. M. Goyal, I. J. Soon, H. S. Joo, and J. E. Collins 1996. Structural polypeptides of the American (VR-2332) strain of porcine reproductive and respiratory syndrome virus. Arch. Virol. 141:1357-1365.
3. de Lima, M., A. K. Pattnaik, E. F. Flores, and F. A. Osorio. 2006. Serologic marker candidates identified among B-cell linear epitopes of Nsp2 and structural proteins of a North American strain of porcine reproductive and respiratory syndrome virus. Virology 353:410-421.
4. Dea, S., L. Wilson, D. Therrien, and E. Cornaglia. 2000. Competitive ELISA for detection of antibodies to porcine reproductive and respiratory syndrome virus (isolate ATCC VR-2332) in North America and experimental reproduction of the disease in gnotobiotic pigs. J. Vet. Diagn. Investig. 4:117-126.
5. den Boon, J. A., Snijder, E. J., Chirnside, E. D., de Vries, A. A., Horzinek, M. C., and Spaan, W. J. (1991). Equine arteritis virus is not a togavirus but belongs to the coronavirus-like superfamily. J Virol 65: 2910-20.
6. den Boon, J. A., K. S. Faaberg, A. L. Meulenberg, P. G. Wassenaar, A. Plagemann, E. Gorbalenya, and E. A. Snijder. 1995. Processing and evolution of the N-terminal region of the arterivirus replicase ORF1a protein: identification of two papain-like cysteine proteases. J. Virol. 69:4500-4505.
7. Denac, H., C. Moser, J. D. Tratschin, and M. A. Hofmann. 1997. An indirect ELISA for the detection of antibodies against porcine reproductive and respiratory syndrome virus using recombinant nucleocapsid protein as antigen. J. Virol. Methods. 65:169-181.
8. Fang, Y., K. Dal-Young, S. Ropp, P. Steen, J. Christopher-Hennings, E. A. Nelson, and R. R. R. Rowland. 2004. Heterogeneity in Nsp2 of European-like porcine reproductive and respiratory syndrome viruses isolated in the United States. Virus Res. 100:229-235.
9. Fang, Y., P. Schneider, W. P. Zhang, K. S. Faaberg, E. A. Nelson, and R. R. R. Rowland. 2007. Diversity and evolution of a newly emerged North American Type 1 porcine arterivirus: analysis of isolates collected between 1999 and 2004. Arch. Virol. 152:1009-1017.
10. Ferrin, N. H., Y. Fang, C. R. Johnson, M. P. Murtaugh, D. D. Polson, M. Torremorell, M. L. Gramer, and E. A. Nelson. 2004. Validation of a blocking enzyme-linked immunosorbent assay for detection of antibodies against porcine reproductive and respiratory syndrome virus. Clin. Diagn. Lab. Immunol. 11:503-514.
11. Gao, Z. Q., X. Guo, and H. C. Yang. 2004. Genomic characterization of two Chinese isolates of porcine respiratory and reproductive syndrome virus. Arch. Virol. 149:1341-1351.
12. Godney, E. K., Chen, L., Kumar, S. N., Methyen, S. L., Koonvin, E. V., and Brinton, M. A. (1993). Complete genomic sequence and Phylogenetic analysis of the lactate dehydrogenase-elevating virus (LDV). Virology 194: 585-596.
13. Gorbalenya, A. E., V. M. Blinov, A. P. Donchenko, and E. V. Koonin. 1989. An NTP binding motif is the most conserved sequence in a highly diverged monophyletic group of proteins involved in positive strand RNA viral replication. J. Mol. Evol. 28:256-268.
14. Han, J., G. Liu, Y. Wang, and K. S. Faaberg. 2007. Identification of nonessential regions of the nsp2 replicase protein of porcine reproductive and respiratory syndrome virus strain VR-2332 for replication in cell culture. J. Virol. 81:9878-9890.
15. Johnson, C. R., Y. Wanquin, and M. P. Murtaugh. 2007. Cross-reactive antibody responses to nsp1 and nsp2 of porcine reproductive and respiratory syndrome virus. J. Gen. Virol. 88:1184-1195.
16. Kairisto, V., and A. Poola. 1995. Software for illustrative presentation of basic clinical characteristics of laboratory tests—GraphRoc for Windows. Scand. J. Clin. Lab. Investig. Suppl. 222:43-60.
17. Kim, H. S., J. Kwang, K. J. Yoon, H. S. Joo and M. L. Frey. 1993. Enhanced replication of porcine reproductive and respiratory syndrome (PRRS) virus in a homogeneous subpopulation of MA-104 cell line. Arch. Virol. 133, 477-483.
18. Lawson, S., Y. Fang, R. R. R. Rowland, C. Christopher-Hennings, and E. A. Nelson. 2005. Experimental infection of pigs with European-like (Type 1) PRRSV isolates of US origin. In The 86th Annual Meeting of the Conference of Research Workers in Animal Diseases, abstract no. 99, 4-6 Dec. 2005, St Louis, Mo., USA.
19. Loemba, H. D., Mounir, S., Mardassi, H., Archambault, D., and Dea, S. (2002). Kinetics of humoral immune response to the major structural proteins of the porcine reproductive and respiratory syndrome virus. Arch Virol 141: 751-761.
20. Mardassi, H., B. Massive, and S. Dea. 1996. Intracellular synthesis, processing, and transport of proteins encoded by ORFs 5 to 7 of porcine reproductive and respiratory syndrome virus. Virology 221:98-112.
21. Meng, X. J., P. S. Paul, P. G. Halbur, and M. A. Lum. 1995. Phylogenetic analyses of the putative M (ORF 6) and N(ORF 7) genes of porcine reproductive and respiratory syndrome virus (PRRSV): implication for the existence of two genotypes of PRRSV in the U.S.A. and Europe. Arch. Virol. 140:745-755.
22. Meulenberg, J. J. M., A. Petersen-den Besten, E. P. de Kluyver, R. J. M. Moormann, W. M. M. Schaaper, and G. Wensvoort. 1995. Characterization of proteins encoded by ORFs 2 to 7 of Lelystad virus. Virology 206:155-163.
23. Meulenberg, J. J. M., and A. Petersen-den Besten. 1996. Identification and characterization of a sixth structural protein of Lelystad virus: the glycoprotein GP2 encoded by ORF2 is incorporated in virus particles. Virology 225:44-51.
24. Mounir, S., H. Mardassi, and S. Dea. 1995. Identification and characterization of the porcine reproductive and respiratory syndrome virus ORFs 7, 5, and 4 products. Adv. Exp. Med. Biol. 80:317-320.
25. Mulupuri, P., J. J. Zimmerman, J. Hermann, C. R. Johnson, J. P. Cano, W. Yu, S. A. Dee, and M. P. Murtaugh. 2008. Antigen-specific B-cell responses to porcine reproductive and respiratory syndrome virus infection J. Virol. 82:358-70.
26. Murtaugh, M. P., Zhengguo, X., and Zuckermann, F. (2002) Immunological Responses of Swine to Porcine Reproductive and Respiratory Syndrome Virus Infection. Viral Immunology 15(4): 533-547.
27. Nelsen, C. J., M. P. Murtaugh, and K. S. Faaberg. 1999. Porcine reproductive and respiratory syndrome virus comparison: divergent evolution on two continents. J. Virol. 73:270-280.
28. Oleksiewicz, M. B., A. B\acute{ø}tner, P. Toft, P. Normann, and T. Storgarrd. 2001a. Epitope mapping porcine reproductive and respiratory syndrome virus by phage display: the nsp2 fragment of the replicase polyprotein contains a cluster of B-cell epitopes. J. Virol. 75:3277-3290.
29. Oleksiewicz, M. B., A. B\acute{ø}tner, and P. Normann. 2001b. Semen form boars infected with porcine reproductive and respiratory syndrome virus (PRRSV) contains antibodies against structural as well as non-structural viral proteins. Vet. Micro. 81:109-125.
30. Oleksiewicz, M. B., A. B\acute{ø}tner, and P. Normann. 2002. Porcine B-cells recognize epitopes that are conserved between the structural proteins of American- and European-type porcine reproductive and respiratory syndrome virus. J. Gen. Virol. 83:1407-1418.
31. Osorio, F. A., Galeota, J. A., Nelson, E., Brodersen, B., Doster, A., Wills, R., Zuckermann, F., and Laegreid, W. W. (2002). Passive transfer of virus-specific antibodies confers protection against reproductive failure induced by a virulent strain of porcine reproductive and respiratory syndrome virus and establishes sterilizing immunity. Virology 302(1):9-20.
32. Ran, Z. G., X. Y. Chen, X. Guo, X. N. Ge, K. J. Yoon, and H. C. Yang. 2008. Recovery of viable porcine reproductive and respiratory syndrome virus from an infectious clone containing a partial deletion within the Nsp2-encoding region. Arch. Virol. 153:899-907.
33. Ropp, S. L., C. E. Wees, Y. Fang, E. A. Nelson, K. D. Rossow, M. Bien, B. Arndt, S. Preszler, P. Steen, J. Christopher-Hennings, J. E. Collins, D. A. Benfield, and K. S. Faaberg. 2004. Characterization of emerging European-like porcine reproductive and respiratory syndrome virus isolates in the United States. J. Virol. 78:3684-3703.
34. Seuberlich, T., J. D. Tratschin, B. Thur, and M. A. Hofmann. 2002. Nucleocapsid protein-based enzyme-linked immunosorbent assay for detection and differentiation of antibodies against European and North American Porcine Reproductive and Respiratory Syndrome Virus. Clin. Diag. Lab. Immunol. 9:1183-1191.
35. Shen, S., J. Kwang, W. Liu, and D. X. Liu. 2000. Determination of the complete nucleotide sequence of a vaccine strain of porcine reproductive and respiratory syndrome virus and identification of the Nsp2 gene with a unique insertion. Arch. Virol. 145:871-883.
36. Snijder, E. J., and J. M. Meulenberg. 1998. The molecular biology of arteriviruses. J. Gen. Virol. 79:961-979.
37. Snijder, E. J., and W. J. Spaan. 2007. Arteriviruses. In Fields Virology.; Knipe, D. M., and P. M. Howley. Lippincott Williams & Wilkins. Vol. 1, ed. 5, pgs 1337-1355.
38. Tian, K., X. Yu, T. Zhao, Y. Feng, Z. Cao, C. Wang, and other authors. 2007. Emergence of fatal PRRSV variants: unparalleled outbreaks of atypical PRRS in China and molecular dissection of the unique hallmark. PLoS ONE 2:526.
39. Van Dinten, L. C., A. L. Wassenaar, A. E. Gorbalenya, W. J. Spaan, and E. A. Snijder. 1996. Processing of the equine arteritis virus replicase ORF1b protein: identification of cleavage products containing the putative viral polymerase and helicase domains. J. Virol. 70:6625-6633.
40. Wootton, S. K., Nelson E. A., and Yoo, D. (1998). Antigenic structure of the nucleocapsid protein of porcine reproductive and respiratory syndrome virus. Clin Diag Lab Immun: 773-779.
41. Wu, W. H., Y. Fang, R. Farwell, M. Steffen-Bien, R. R. R. Rowland, J. Christopher-Hennings, and E. A. Nelson. 2001. A 10-kDa structural protein of porcine reproductive and respiratory syndrome virus encoded by ORF 2b. Virology 287:183-191.
42. Wu, W. H., Y. Fang, R. R. R. Rowland, S. R. Lawson, J. Christopher-Hennings, K. J. Yoon, and E. A. Nelson. 2005. The 2b protein as a minor structural component of PRRSV. Virus Res. 114:177-181.

What is claimed is:

1. An in vitro method of detecting, identifying or quantifying a humoral immune response to porcine reproductive respiratory syndrome virus (PRRSV), comprising:
contacting a biological sample with nonstructural protein 7 (nsp7) antigen derived from PRRSV to effect binding of antibodies in the biological sample to the nsp7 antigen;
introducing a detection system for detecting the presence or absence of a complex between the nsp7 antigen and an antibody; and
determining, quantitatively and/or qualitatively, the presence or absence of a complex between the nsp7 antigen and an antibody, wherein the presence of a complex indicates an immune response to PRRSV.

2. The method according to claim 1 wherein said nsp7 antigen is derived from Type 1 (European) PRRSV.

3. The method according to claim 1 wherein said nsp7 antigen is derived from Type 2 (North American) PRRSV.

4. The method according to claim 1 wherein the nsp7 antigen comprises both Type 1 (European) PRRSV and Type 2 (North American) PRRSV antigens.

5. The method according to claim 1 wherein the nsp7 antigen comprises a peptide sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:43, or a combination thereof.

6. The method according to claim 1 wherein said nsp7 antigen comprises SEQ ID NO: 43 and an nsp7 antigen derived from Type 2 (North American) PRRSV.

7. The method according to claim 5 wherein said nsp7 antigen comprises an epitope obtained from a peptide sequence selected from the group consisting of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or a combination thereof.

8. The method according to claim 1 further comprising comparing the presence or absence of a complex between the nsp7 antigen and an antibody from said method with a determination of an immunological response using an IDESS enzyme-linked immunosorbent assay (ELISA) analysis performed on the same biological sample.

9. The method according to claim 1, wherein detecting the presence or absence of a complex between the nsp7 antigen and an antibody comprises the use of an enzyme-linked immunosorbent assay (ELISA).

10. The method according to claim 1, wherein the detection system comprises an antiporcine antibody covalently or noncovalently attached to a detectible marker.

11. The method according to claim 10, wherein the detectible marker is selected from the group consisting of a radioisotope, an enzyme, a fluorescent molecule, a magnetic particle, an electron opaque substance or a combination thereof.

12. The method according to claim 1, further comprising contacting the biological sample to a first nsp7 antigen derived from a Type 1 (European) PRRSV and a second nsp7 antigen derived from a Type 2 (North American) PRRSV; and
determining the whether the biological sample was obtained from an animal having an immune reaction to Type 1 or Type 2 PRRSV

13. The method according to claim 1, wherein the biological sample a porcine biological sample selected from the group consisting of blood, serum, plasma, tears, mucous, and nasal secretions.

14. A kit for conducting the method according to claim 1, the kit comprising:
a solid support; and
at least one nonstructural protein 7 antigenic reagent for contacting with a biological sample.

15. The kit according to claim 14, further comprising a detection system for detecting the presence of antibodies in a biological sample.

16. The kit according to claim 14, wherein said antigenic reagent comprises nonstructural protein 7, and/or epitopes thereof, derived from a Type 1 (European) PRRSV and a nsp7 antigen derived from a Type 2 (North American) PRRSV.

17. The kit according to claim 14, wherein said nonstructural protein 7 is an epitope derived from Type 1 (European) PRRSV.

18. The kit according to claim 14, wherein said nonstructural protein 7 is an epitope derived from Type 2 (North American) PRRSV.

19. The kit according to claim 15, wherein a detection system comprises a detection antibody covalently or noncovalently attached to a detectable marker.

20. The kit according to claim 19, wherein the detectable marker is selected from the group consisting of a radioisotope, an enzyme, a fluorescent molecule, a magnetic particle, an electron opaque substance or a combination thereof.


SEQUENCE LISTING (43 sequences)

SEQ ID NO: 1 - protein, 861 aa, Porcine reproductive and respiratory syndrome virus
AAGKRARAKRAAKSEKDSAPTPKVALPVPTCGITTYSPPTDGSCGWHVLAAIMNRMINGDFTSPLTQYNRPEDDWASDYDLVQAIQCLRLPATVVRNRACPNAKYLIKLNGVHWEVEVRSGMAPRSLSRECVVGVCSEGCVAPPYPADGLPKRALEALASAYRLPSDCVSSGIADFLANPPPQEFWTLDKMLTSPSPERSGFSSLYKLLLEVVPQKCGATEGAFIYAVERMLKDCPSSKQAMALLAKIKVPSSKAPSVSLDECFPTDVLADFEPASQERPQSSGAAVVLCSPDAKEFEEAAPEEVQESGHKAVHSALLAEGPNNEQVQVVAGEQLKLGGCGLAVGNAHEGALVSAGLINLVGGNLSPSDPMKENMLNSREDEPLDLSQPAPASTTTLVREQTPDNPGSDAGALPVTVREFVPTGPILCHVEHCGTESGDSSSPLDLSDAQTLDQPLNLSLAAWPVRATASDPGWVHGRREPVFVKPRNAFSDGDSALQFGELSESSSVIEFDRTKDAPVVDAPVDLTTSNEALSVVDPFEFAELKRPRFSAQALIDRGGPLADVHAKIKNRVYEQCLQACEPGSRATPATREWLDKMWDRVDMKTWRCTSQFQAGRILASLKFLPDMIQDTPPPVPRKNRASDNAGLKQLVAQWDRKLSVTPPPKPVGPVLDQIVPPPTDIQQEDVTPSDGPPHAPDFPSRVSTGGSWKGLMLSGTRLAGSISQRLMTWVFEVFSHLPAFMLTLFSPRGSMAPGDWLFAGVVLLALLLCRSYPILGCLPLLGVFSGSLRRVRLGVFGSWMAFAVFLFSTPSNPVGSSCDHDSPECHAELLALEQRQLWEPVRGLVVGPSGLLCVILGKLLG

SEQ ID NO: 2 - protein, 860 aa, Porcine reproductive and respiratory syndrome virus
AAGKRARAKRAAKSEKGSAPTPKVVLPVPTCGVTTYSPPTDGSCGWHVLAAIMNRMINGDFTSPLTQYNRPEDDWASDFDLIQAIQCLQLPATVVRNRACPNAKYLIKLNGVHWEVEVRSGMAPRSLSRECVVGVCSEGCVAPPYPADGLPKRALEALASAYRLPSDCVSSGIADFLANPPPQELWTLDRMLTSPSPERSGFSSLYKLLLEVVPKKCGATEGAFIYAVERMLKDCPSSKQAMTLLAKIKVPSSKAPSVSLDECFPTDVSADFEPAFQERPQSSGAADVLCSLGAKEFEGAAPGEVQEGGDEAVRSAPLAVDLNDEHARVVVGEQLKLGGCGLTVGNAHEGVLAPTGPINLVSGNLSPSDSMRGNMLNSREDEPLDLSLPAPVATTTLVREQTPDNPGSDAGPLPVTVRELAPTGPILRHVEHCGTESGESSSPLNCSDAQTQDQPLNLSLAAWPVRVAASDPGWVYGRREPVFVKPRKAFSDGDSALQFGELSESSSVVESDRMNDAPVVDAPVDLTTSNEALSVVDLFEFAELKRPRFSAQALIDRGGPLADVHAKIKNQVYERCLQACEPGSRATPATREWLDKMWERVDMKTWRCTSQFQAGRILASLKFLPDMIQDTPPPVPRKNRASDNAGLKQLVAQWDRKLSMTSPQKPVGSVLDQTAFPPMDSQQENVTPSDGPPHAPDCPSRVSMGGGWKGLVFSSTRFAGSSQRLMTWVFEVVSHLPAFMLTLFSPRGSMAPGDWLFAGVVLLSLLLCHSYPIFGCLPLLGVFSGSLRRVRLGVFGSWMAFAVFLFSTPSNPVGSSCDHDSPECHAELLALEQRQLWEPVRGLVVGPSGLLCVILGKLLG

SEQ ID NO: 3 - protein, 855 aa, Porcine reproductive and respiratory syndrome virus
AAGKRARAKRAVKGEKDSAPTPEVALSVPTCGTTTYSPPTDGSCGWHVLAAIMNRMINGDFTSPLAPHNRPEDDWASDYDLVQAIQCLQLPATVVRNRACPNAKYLIKLNGVHWEVEARSGMAPRSLSRECVVGVCSEGCVAPPYPADGLPKRALEALASAYRLPSDCVSSGIADFLANPPPQEFWTLDKMLTSPSPERSGFSSLYKLLLEVVPRKCGTTEGAFTYAVERMLKDCPSSKQAMALLAKIKVPSSKAPSVSLDECFPADAPADFEPAFQEKPRSSGAAIALKGFGEAAPEEVLESSRKTVHSALRAEGSNEQAQAVAGEQLKLGGCGSAVGNAHGGVPVLAGLINLVGGNFSPPDSMKGNTLNSLEDEPLDLSRPALAVTTSLVKEQTSGNPGLGAGALPVTVREFVPTGPALRHVEHCGAESDDSSSPLDLSDTQTLDQPLNLSLAAWPVKATASDPGWVHGRCEPVFVKPRKAFSDGDSVLQLGELSESSSVIEFDRTKDAPVVDAPVDLTTSNEAFSVCDPFEFAELKRPRFSAQALIDRGGPLADVHAKIKNRVYEQCLKACEPGSRATPATKEWLDKMWDRVDMKTWRCTSQFQAGRILASLKFLPDMIQDTPPPVPRKNRASDNAGLKQLVAQWDRKFSVTPPPKLAGPVLDQTVLPPTDAQREDATPSDELPHAPHLLSRVSMGGSWKGLIRSGTRLAGSVSQHLMTWVFEVFSHLPAFMLTLFSPRGSMAPGDWLFAGVVLLTLLLCRTYPILGCLPLLGVFSGSLRRVRLGVFGSWMAFAVFLFSVPSNPVGSSCDHDSPECHAELLTLEQRQLWEPVRGLVVGPSGLLCVILGKLLG

SEQ ID NO: 4 - protein, 844 aa, Porcine reproductive and respiratory syndrome virus
AAGKRARAKRATKGEKDSALAPIVPLPVPTCGITTYSPPTDGSCGWHVLAAIVNRMINGDFTSPLPQYNRPEDDWASDYDLAQAIQCLQLPATVVRNRACPNAKYLIKLNGVHWEVEVRSGMAPRSLSRECVVGVCSEGCVAPPYPADGLPKRALEALASAYRLPSDCVSSGIADFLADPPPQEFWTLDKMLTSPSPERSGFSSLYKLLLEVVPQKCGATEGAFVYAVERMLKDCPSPEQAMALLAKIKVPSSKAPSVSLDECFPAGVPADFEPAFQERPRSPGAAVALCSPDAKGFEGTASEEAQESGHKAVHAVPLAEGPNNEQVQVVAGEQLELGGCGLAIGSAQSSSDSKRENMHNSREDEPLDLSHPAPAATTTLVGEQTPDNPGSDASALPIAVGEFVPTGPILRHVEHCGTESGDSSSPLDLSFAQTLDQPLDLSLAAWPVKATASDPGWVRGRCEPVFVKPRKAFSDGDSALQFGELSESSSVIEFDQTKDTLVADAPVDLTTSNEALSAVDPSEFVELRRPRHSAQALIDRGGPLADVHAKIKNRVYEQCLQACEPGSRATPATREWLDKMWDRVDMKTWRCTSQFQAGRILASLKFLPDMIQDTPPPVPRKNRASDNAGLKQLVARWDKKLSVTPPPKSAGLVLDQTVPPPTDIQQEDATPSDGLSHASDFSSRVSTSWSWKGLMLSGTRLAGSAGQRLMTWVFEVYSHLPAFILTLFSPRGSMAPGDWLFAGVVLLALLLCRSYPILGCLPLLGVFSGSLRRVRLGVFGSWMAFAVFLFSTPSNPVGSSCDHDSPECHAELLALEQRQLWEPVRGLVVGPSGLLCVILGKLLG

SEQ ID NO: 5 - protein, 980 aa, Porcine reproductive and respiratory syndrome virus
AGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKIDLYLRGATNLEECLARLEKARPPRVIDTSFDWDVVLPGVEAATQTIKLPQVNQCRALVPVVTQKSLDNNSVPLTAFSLANYYYRAQGDEVRHRERLTAVLSKLEKVVREEYGLMPTEPGPRPTLPRGLDELKDQMEEDLLKLANAQTTSDMMAWAVEQVDLKTWVKNYPRWTPPPPPPKVQPRKTKPVKSLPERKPVPAPRRKVGSDCGSPVSLGGDVPNSWEDLAVSSPFDLPTPPEPATPSSELVIVSSPQCIFRPATPLSEPAPIPAPRGTVSRPVTPLSEPIPVPAPRRKFQQVKRLSSAAAIPPYQDEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQAICTLDGRLKFLPKMILETPPPYPCEFVMMPHTPAPSVGAESDLTIGSVATEDVPRILEKIENVGEMANQGPLAFSEDKPVDDQLVNDPRISSRRPDESTSAPSAGTGGAGSFTDLPPSDGADADGGGPFRTVKRKAERLFDQLSRQVFDLVSHLPVFFSRLFYPGGGYSPGDWGFAAFTLLCLFLCYSYPAFGIAPLLGVFSGSSRRVRMGVFGCWLAFAVGLFKPVSDPVGAACEFDSPECRNILHSFELLKPWDPVRSLVVGPVGLGLAILGRLLG

SEQ ID NO: 6 - protein, 980 aa, Porcine reproductive and respiratory syndrome virus
AGKRARKARSGMTTTVAHRALPAREIQQAKKHEDAGADKAVHLRHYSPPADGNCGWHCISAIANRMVNSKFETTLPERVRPPDDWATDEDLVNTIQILKLPAALDRNGACVGAKYVLKLEGEHWTVSVTLGMSPSLLPLECVQGCCEHKGGLGPPDAVEVFGFDPACLDRLAEVMHLPSSVIPAALAEMSGDPNCPASPVTTVWTVSQFFARHRGENHPDQVRLGKIISLCQVVEECCCHQNKTNRATPEEVAARIDQYLHGATSLEECLIRLERVCPPSAADTFFDWNVVLPGVGASTQTTKQLHVNQCRALVPVVTQEPLDKDSVPLTAFSLSNCYYPAQGDEVRHRERLNSVLSKLEGVVREEYGLTPTEPGPRPALPNGLVELKDQMEEDLLKLVNAQATSEMMAWAAEQVDLKAWVKNYPRWTPPPPPPRVQPRKTKSVKSLPGNKPVPAPRRKVRSDCGSPILMGDNVPDGREDLTVGGPLDLSTPSEPMTPLSEPALMPALQYISRPVTSLSVLAPVPAPRRTVSRPVTPLSEPIFVSAPRHKFQQVEEANLAATTLTHQDEPLDLSASSQTEYEASPLTPLQNMGILEVGGQEAEEVLSEISDTLNDINPAPVSSSSSLSSVKITRPKHSAQAIIDSGGPCSGHLRREKEACLSIMREACDAAKLSDPATQEWLSRMWDRVDMLTWRNTSAYQAFRELDGRFEFLPKMILETPPPYPCEFVMLPHTPAPSVGAESDLTIGSVATEDVPRILGKIENAGEMPNQGLLTSFGEEPVCDQPVKDSWMSSRGFDESTTAPSAGTGGADLPTDLPPSDGLDADEWGPLRTVRKKAERLFDQLSRQVFNLVSHLPVFFSHLFKSDSGYSPGDWGFAAFTLFCLFLCYSYPFFGFVPLLGVFSGSSRRVRMGVFGCWLAFAVGLFKPVSDPVGTACEFDSPECRNVLHSFELLKPWDPVRSLVVGPVGLGLAILGRLLG

SEQ ID NO: 7 - protein, 980 aa, Porcine reproductive and respiratory syndrome virus
AGKRARKARSCATATVAGRALSVRETRQAKEHEVAGANKAEHLKHYSPPAEGNCGWHCISAIANRMVNSKFETTLPERVRPPDDWATDEDLVNAIQILRLPAALDRNGACTSAKYVLKLEGEHWTVTVTPGMSPSLLPLECVQGCCGHKGGLGSPDAVEVSGFDPACLDRLAEVMHLPSSAIPAALAEMSGDSDRSASPVTTVWTVSQFFARHSGGNHPDQVRLGKIISLCQVIEDCCCSQNKTNRVTPEEVAAKFDLYLRGATNLEECLARLEKARPPRVIDTFFDWDVVLPGVEAATQTIKLPQVNQCRALVPVVTQKSLDNNSVPLTAFSLANYYYRAQGDEVRHRERLTAVLSKLEKVVREEYGLMPTKPGPRPTLPRGLDELKDQMEEDLLKLANAQTTSDMMAWAAEQVDLKTWVKNYPRWTPPPPSPKVQLRKTKPVKSLPKRKPVPAPRRKVGSDCGSPVSLGGDVPNSWEDLAVSSPFDLPTPPEPAIPSSELVIVSSPQCIFRPATPLSEPAPIPAPRGTVSRPVTPLSEPIPVPAPRRKFQQVKRLSSAAAIPPYQNEPLDLSASSQTEYEASPPAPPQSGGVLGVEGHEAEETLSEISDMSGNIKPASVSSSSSLSSVRITRPKYSAQAIIDSGGPCSGHLQEVKETCLSVMREACDATKLDDPATQEWLSRMWDRVDMLTWRNTSVYQAICTLDGRLKFLPKMILETPPPYPCEFVMMPHTPAPSVGAESDLTIGSVTTEDVPRILEKIGNVGEMANQGPLAFSEDKPVDDQLVNDPRISSRRPDESTSAPSAGTGGAGSFTDLPPSDGADADGGGPFRTVKRKAERLFDQLSRQVFDLVSHLPVFFSRLFYPGGGYSPGDWGFAAFTLLCLFLCYSYPAFGIAPLLGVFSGSSRRVRMGVFGCWLAFAVGLFKPVSDPVGAACEFDSPECRNILHSFELLKPWDPVRSLVVGPVGLGLAILGRLLG

SEQ ID NO: 8 - protein, 980 aa, Porcine reproductive and respiratory syndrome virus
AGKRARKARSGATTMVAHRASSAHEIQQATKHEGAGANKAEHLKLYSPPAEGNCGWHCISAIVNRMVNSNFETTLPERVRPPDDWATDEDLVNTIQILKLPAALDRNGACGGAKYVLKLEGEHWTVSVNPGMSPSLLPLECVQGCCEHKGGLGPPDAVEVSGFDPACLDRLLQVMHLPSSTIPAALAELSDDSNRPVSPAAATWTVSQSYARHRGGNHHDQVCLGKIISLCQVIEDCCCHQNKTNRATPEEVAAKIDQYLRGATSLEECLAKLERVCPPGAADTSFDWNVVLPGVEAAHQTEKQLHVNPCRTLVPPVTQEPLGKDSVPLTAFSLSNCYYPAQGNEVRHRERLNSVLSKLEEVVLEEYGLMSTGLGPRPVLPSGLVELKDQMEEDLLKLVNTQATSEMMAWAAEQVDLKAWVKSYPRWTPPPPPPRVQPRKTKSVKSLPEDKPVPAPRRKVRSGCGSPVLMGDNVPNGREDLTVGGPLNFPTPSEPMTPMSEPVLTPALQRVPKLMTPLDGSAPVPAPRRTVSRPMTPLSEPIFLSAPRHKFQQVEEANPATTTLTHQNEPLDLSASSQTEYEASPLASSQNMSILEAGGQEAEEVLSEISDILNDTSPAPVSSSSSLSSVKITRPKYSAQAIIDSGGPCSGHLQKVKEACLSIMREACDASKLSDPATQEWLSRMWDRVDMLTWRNTSAYQFRTLNDGRFEFLPKMILETPPPHPCGFVMLPHTPAPSVSAESDLTIGSVATEDVPRILGKIGDTGELLNQGPSAPFKGGPVCDQPAKNSRMSPRESDESIIAPPADTGGAGSFTDLPSSDSVDANGGGPLRTVKTKAGRLLDQLSCQVFSLVSHLPVFFSHLFKSDSGYSPGDWGFAAFTLFCLFLCYSYPFFGFAPLLGVFSGSSRRVRMGVFGCWLAFAVGLFKPVSDPVGTACEFDSPECRNVLHSFELLKPWDPVRSLVVGPVGLGLAILGRLLG

SEQ ID NO: 9 - protein, 259 aa, Porcine reproductive and respiratory syndrome virus
SLTGALAMRLNDEDLDFLTKWTDFKCFVSASNMRNAAGQFIEAAYAKALRVELAQLVQVDKVRGTLAKLEAFADTVAPQLSPGDIVVALGHTPVGSIFDLKVGSTKHTLQAIETRVLAGSKMTVARVVDPTPTPPPAPVPIPLPPKVLENGPNAWGDEDRLNKKKRRRMEALGIYVMGGKKYQKFWDKNSGDVFYEEVHNNTDEWECLRVGDPADFDPEKGTLCGHVTIENKAYHVYTSPSGKKFLVPVNPENGRVQWE

SEQ ID NO: 10 - protein, 259 aa, Porcine reproductive and respiratory syndrome virus
SLTGALAMRLNDEDLDFLMKWTDFKCFVSASNMRNAAGQFIEAAYAKALRVELAQLVQVDKVRGTLAKLEAFADTVAPQLSPGDIVVALGHTPVGSIFDLKVGSTKHTLQAIETRVLAGSKMTVARVVDPTPTPPPAPVPIPLPPKVLENGPNAWGDEDRLNKKKRRRMEALGIYVMGGKKYQKFWDKNSGDVFYEEVHNNTDEWECLRVGDPADFDPEKGTLCGHVTIENKAYHVYTSPSGKKFLVPVNPENGRVQWE

SEQ ID NO: 11 - protein, 259 aa, Porcine reproductive and respiratory syndrome virus; identical to SEQ ID NO: 10

SEQ ID NO: 12 - protein, 259 aa, Porcine reproductive and respiratory syndrome virus
SLTGALAMRLNDEDLDFLMKWTDFKCFVSASNMRNAAGQFIEAAYAKALRVELAQLVQVDKVRGTLAKLEAFADTVAPQLSPGDIVVALRHTPVGSIFDLKVGITKHTLQAIETRVLAGSKMTVARVVDPTPTPPPAPVPIPLPPKVLENGPNAWGDEDRLNKKKRRRMEALGIYVMGGKKYQKFWDKNSGDVFYEEVHNNTDEWECLRVGDPADFDPEKGTLCGHVTIENKAYHVYTSPSGKKFLVPVNPENGRVQWE

SEQ ID NO: 13 - protein, 259 aa, Porcine reproductive and respiratory syndrome virus
SLTGALAMRLNDEDLDFLMKWTDFKCFVSASNMRNAAGQFIEAAYAKALRVELAQLVQVDKVRGVLAKLEAFADTVAPQLSPGDIVVALGHTPVGSIFDLKVGSTKHTLQAIETRVLAGSKMTVARVVDPTPTPPPAPVPIPLPPKVLENGPNAWGDEDRLNKKKRRRMEALGIYVMGGKKYQKFWDKNSGDVFYEEVHNNTDEWECLRVGDPADFDPEKGTLCGHVTIENKAYHVYISPSGKKFLVPVNPENGRVQWE

SEQ ID NO: 14 - protein, 259 aa, Porcine reproductive and respiratory syndrome virus
SLTGALAMRLNDEDLDFLTKWTDFKCFVSASNMRNAAGQFIEAAYAKALRVELAQLVQVDKVRGTLAKLEAFADTVAPQLSPGDIVVALGHTPVGSIFDLKVGNTKHTLQAIETRVLAGSKMTVARVVDPTPTLPPAPVPIPLPPKVLENGPNAWGDEDRLNKKRRRRMEALGIYVMGGKKYQKFWDKNSGDVFYEEVHNNTDEWECLRVGDPADFDPEKGTLCGHVTIEDRAYHVYTSPSGKKFLVPVNPENGRVQWE

SEQ ID NO: 15 - protein, 259 aa, Porcine reproductive and respiratory syndrome virus
SLTGALAMRLTDEDLDFLTKWTDFKCFVSASNMRNAAGQFIEAAYAKALRVILAQLVQVDKVRGTMAKLEAFADTVAPQLSPGDIVVALGHTPVGSIFDLKVGSTKHTLQAIETRVLAGSKMTVARVVDPTPAPPPVPVPIPLPPKVLENGPNAWGDEDRLNKKKRRRMEAVGIFVMDGKKYQKFWDKNSGDVFYEEVHISTDEWECLRTGDPVDFDPETGIQCGHITIEDKVYNVFTSPSGRRFLVPANPENRRAQWE

SEQ ID NO: 16 - protein, 259 aa, Porcine reproductive and respiratory syndrome virus
SLTGALAMKLSDEDLDFLTKLTDFKCFVSASNMRNAAGQFIEAAYAKILRVELAQLVQVDKVRGVLAKLEAFADTVAPHLSPGDIVVVLGHTPVGSIFDLKIGNAKHTLQAIETRVLAGSRMTVARVVDPTPAPPPVPVPVPLPPKVLENGPSAWGDEDRLNKKKRRKMEAVGIYVMGGKKYQKFWDKNSGDVFYEEVHDNTDAWECLRADDPADLDPERGTLCGHVTIENRPYHVYASPSGRKFLVPADPENGKAQWE

SEQ ID NO: 17 - protein, 259 aa, Porcine reproductive and respiratory syndrome virus
SLTGALAMRLNDEDLDFLTKWTDFKCFVSASNMRNAAGQFIEAAYAKALRVILAQLVQVDKVRGTLAKLEAFADTVAPQLSPGDIVVALGHTPVGSIFDLKVGGTKHTLQVIETRVLAGSKMTVARVVDPTPTPPPAPVPIPLPPKVLENGPNAWGDGDRLNKKKRRRMETVGIFVMGGKKYQKFWDKNSGDVFYEEVHDNTDAWECLRVGDPADFDPEKGTLCGHTTIEDKDYKVYISPSGKKFLVPVNSESGRAQWE

SEQ ID NO: 18 - protein, 259 aa, Porcine reproductive and respiratory syndrome virus; identical to SEQ ID NO: 13

SEQ ID NO: 19 - protein, 259 aa, Porcine reproductive and respiratory syndrome virus; identical to SEQ ID NO: 15

SEQ ID NO: 20 - DNA, 807 bp, Porcine reproductive and respiratory syndrome virus
tccctgacggctgctctagcttgcaagttgtcgcaggctgaccttgattttttgtccagcttaacgaacttcaagtgctttgtatctgcttcaaacatgaaaaatgctgccggccagtacattgaagcagcttatgccaaggccctgcgccaagagttggcctctctagttcaggttgacaaaatgaaaggaattttgtccaagctcgaggcctttgctgaaacagccaccccgtcccttgacacaggtgacgtggttgttctgcttgggcaacatcctcacggatccatcctcgatattaatgtggggactgaaaggaaaactgtgtccgtgcaagagacccggagcctaggcggctccaaattcagtgtttgcactgtcgtgtccaacacacccgtggacgccttaaccggcatcccactccagacaccaacccctctttttgagaatggtccgcgtcatcgcggtgaggaagacgatcttaaagtcgagaggatgaagaaacactgtgtgtccctcggcttccacaacatcaatggcaaagtttactgcaaaatttgggacaagtctaccggtgacaccttttacaccgatgattcccggtacacccaagaccatgcttttcaggacaggtcagccgactacagagacagggactatgagggtgtgcaaaccgccccccaacagggctttgatccaaagtctgaaacccctgttggcactgtagtgatcggcggtattacgtataacaggtacctgatcaaaggtaaggaggtcctggttcccaagcctgacaactgccttgaa

SEQ ID NO: 21 - DNA, 807 bp, Porcine reproductive and respiratory syndrome virus
tccctaacggctgctttagcttgcaagttgtcacaggctgaccttgattttttgtccagcttaacgaacttcaagtgctttgtatctgcttcaaacatgaaaaatgctgccggccagtacattgaagcagcgtatgccaaggccctgcgccaagagttggcctctctagttcagattgacaaaatgaaaggagttttgtccaagctcgaggcctttgctgaaacagccaccccgtcccttgacataggtgacgtgattgttctgcttgggcaacatcctcacggatccatcctcgatattaatgtggggactgaaaggaaaactgtgtccgtgcaagagacccggagcctaggcggctccaaattcagtgtttgtactgtcgtgtccaacacacccgtggacgccttgaccggcatcccactccagacaccaacccctctttttgagaatggtccgcgtcatcgcagcgaggaagacgatcttaaagtcgagaggatgaagaaacactgtgtatccctcggcttccacaacatcaatggcaaagtttactgcaaaatttgggacaagtctaccggtgacaccttttacacggatgattcccggtacacccaagaccatgcttttcaggacaggtcagccgactacagagacagggactatgagggtgtgcaaaccaccccccaacagggatttgatccaaagtctgaaacccctgttggcactgttgtgatcggcggtattacgtataacaggtatctgatcaaaggtaaggaggttctggtccccaagcctgacaactgccttgaa

SEQ ID NO: 22 - DNA, 807 bp, Porcine reproductive and respiratory syndrome virus
tctctgacggctgctctggcttgcaagttgtcgcaggctgatcttgaatttttgtccagtttaacgaacttcaagtgctttgtgtctgcttcaaatatgaaaaatgccgccggccagtacattgaagcagcttatgccaaggccttgcgccaagagttggcctctctagttcaggttgataaaatgaaaggagttttgtccaagctcgaggcctttgctgaaacagccaccccgtcccttgacacaggtgacgtggttgttttgcttgggcagcatcctcacgggtctatcctcgatattaatgtggggactgaaaggaaaactgtgtccgtgcaagagacccggaacctaggcggctccaaattcagtgtttgcactgtcgtgtccaacacacccgtggacgccttaaccggcatcccactccggacaccaacccctctttttgagaatggtccgcgtcatcgcggtgaggaagacgatctcaaagtcgagaggatgaagaaacactgtgtatccctcggcttccacaacatcaatggcaaagtctactgtaagatctgggataagtctaccggtgacaccttttacaccgacgattcccggtatacccacgaccatgcttttcaggacaggtcagccgactacagagacagggactacgaaggtgtgcaaaccgccccccaacaaggctttgatccaaagtctgaaacccctgttggcactgtagtgatcggcggtatcacgtataacaggtacctgattagaggtaaggaggtcctggtccccaagcctgacaactgccctgaa

SEQ ID NO: 23 - DNA, 807 bp, Porcine reproductive and respiratory syndrome virus
tccctgacggctgctctagcctgcaagttgtcgcaggctgatcttgacttcttgttcagcttgacgaacctcaagtgcgttgtatctgcttccaacatgaaaaatgctgctggccagtacattgaagcagctaatgctaaggccctgcgccaagagttggcttctctggttcaagttgacaaattgaaaggaattttgtccaagctcgaggcctttgctgaaacagccaccccgtcccctgacacaggtgacgtggttgttctgccttggcaacatcctcacggatccatcctcgatattaatgtggggactgaaaggaaaactgtgtccgtgcaagagacccggagcctaggtgtttccaaattcagtgtttgcactgtcgtgtccaacacacccgtggacgccttaactggcatcccactccagataccggcacctctttttgagaatggtccgcgtcatcgtggcgaggaagatgatcttaaagttgagaggaaggagaaacactgtgtgtctctcggcttccacaacatcaatggcaaagtttactgtaaaatttgggacaagtctaccggtgacacattttacaccgacgattcccggtacacccaagatcatgcttttcaggacaggtcagctgactacagagacagggactatgagggtatgcgaaccgccccccaacagggatttgatccaaagtctgaaacccctgttggcactgttgtgatcggcggtattacgtataacaggtacttgatcaaaggtaaggaggtcctggttcccaagcctgacaactgccttgaa

SEQ ID NO: 24 - DNA, 807 bp, Porcine reproductive and respiratory syndrome virus
tccctgacggctgctctagcttgcaagttgtcgcaggctgaccttgactttttgtccagcttaacgaacttcaagtgctttgtatctgcttcaaacatgaaaaatgctgccggccagtatattgaagcagcgtatgccaaggccctgcgccaagagttggcctccctagttcaggttgacaaaatgaaaggaattttgtctaagcttgaggcctttgctgaaacagccactccgtcccttgacgcaggtgacgtggttgttctgcttgggcaacatcctcacggatccatcctcgatattaatgtggggactgaaaggaaaactgtgtccgtgcaagagacccggagcttaggtggttccaaattcagtgtttgcactgtcgtgtccaacacacccgtggacgccttaactggcatcccactccagacaccaacccctctttttgagaatggtccgcgtcaccgtggtgaggaagacgatcttagagtcgagaggatgaagaaacactgtgtgtccctcggcttccacaacattaatggcaaagtttactgcaaaatttgggacaagtccaccggtgataccttttataccgatgattcccggtacacccaagaccttgcattccaggacaggtcagccgactacagagacagggattatgagggtgtgcaaaccgccccccaacagggctttgatccaaagtctgaaacccctattggcactgtggtgatcggcggtatcacgtataacaggtacctgatcaaaggtaaggaggtcttggttcccaagcctgacaactgccttgaa

SEQ ID NO: 25 - DNA, 807 bp, Porcine reproductive and respiratory syndrome virus
tccctgacggctgctctagcttgcaagttgtcgcaagctgaccttgattttttgtccagcttaacgaacttcaagtgctttgtatctgcttcaaacatgaaaaatgccgctggccagtacattgaagcagcttatgccagggccctacgccaagagttggcctctctagttcaggttgacaagatgaaaggagttttgtccaagctcgaggcctttgctgaaacagccaccccgtcccttgacacaggtgacgtggttgttctgcttgggcaacatcctcacggatccatcctcgatattaatgtggggactgaaaggaaaactgtgtccgtgcaagagacccggagccttggcggctccaaattcagtgtttgcactgttgtgtccaacacacccgtggatgccttaaccggcatcccactccagacaccaacccctctttttgagaatggtccgcgtcatcgcagtgaggaagacgatcttaaagtcgagaggatgaagaaacactgtgtgtccctcggctttcacaacatcaatggcaaagtatactgcaaaatctgggataagtctaccggtgacaccttttacaccgatgattcccggtatacccaagactatgcttttcaggacaggtcagccgactacagagacagggactatgaaggtgtgcaaaccgccccccaacagggctttgatccaaagtctgaaacccctgttggcactgtagtgatcggcggtattacgtataataggtacctgatcaaaggtaaggagatcctggttcccaagcctgacaactgccttgaa

SEQ ID NO: 26 - DNA, 807 bp, Porcine reproductive and respiratory syndrome virus
tccctaacggctgctctagcttgcaagttgtcgcaggctgaccttgattttttgtccagcttaacgaacttcaagtgctttgtatctgcttcaaacatgaaaaatgccgccggccagtacattgaagcagcttatgccaaggccctgcgccaagagttggcctctctagttcaggttgacaaaatgaaaggagttttatccaagctcgaagcctttgctgaaacagccactccgtctcttgacacaggtgacgtggttgttttgcttgggcaacaccctcacggatccatcctcgatattaatgtgggaaatgaaaggaaaactgtgtccgtgcaagagacccggagcctaggcggctccaaattcagtgtttgcactgtcgtttccaacacacccgtggacgccttaaccggcatcccactccagacaccaacccctctttttgagaatggtccgcgccatcgctgtgaggaagacgatcttaaagtcgagaggaaaaaaaaacactgtgtttccctcggctttcacaacatccatggcaaagtttactgcaaaatttgggacaagtctaccggtgacaccttttacaccgatgattctcggtacacccaagaccatgcttttcaggataggtcagccgatcacagagacagagactacgagggcgtgcaaaccgccccccaacagggatttgacccaaagtccgagacccctgttggcactgtagtgatcggcggcattacgtataacaggtacctgatcaaaggtaaggaggtcctggttcgtaagcctgacaactgcctcgaa

SEQ ID NO: 27 - DNA, 807 bp, Porcine reproductive and respiratory syndrome virus
tccctgacggctgccctagcttgcaagttatcgcaggctgaccttgattttttgtccagcttaacgaacttcaagtgctttgtatctgcttcgaacatgaaaaatgctgctggccagtacattgaagcagcttatgccaaggccttgcgccaagagttggcctctctagttcaggtcgataagatgaaaggaattttgtccaagctcgaggcctttgctgaaaccgccaccccgtcccttgatccaggtgacgtggttgttctgcttgggcaacatcctcatggttccatcctcgatattaatgtggggactgaaaggaaaaccgtgtctgtgcaagagacccggagcctaggcggctccaaattcagtgtttgtactgtcgtgtccaacacacctgtggacgccttaaccggcatcccactccagacaccaacccctctttttgagaatggtccgcgtcatcgcagtgaggaagacgacctcaaagtcgagaggatgaagaaacattgtgtatccctcggcttccacaacattaatggaagagtttactgtaaaatttgggacaagtctaccggtgacaccttttataccgatgattcccggtatacccaagatcatgcttttcaggataggtcagccgactacagagacagggactatgaaggtgtgcaagccgctcccccaaagggctttgatccaaagtctgaaacccctgttggcactgtattgaattggcgtgttacgtataacaggtacctgatcaaaggtaaggaggtcctggttcccaaacccgaccactgcctagag

SEQ ID NO: 28 - DNA, 29 bp, Artificial Sequence
  Primer Nsp1F
cgcggatcctctgggatacttgatcggtg

SEQ ID NO: 29 - DNA, 26 bp, Artificial Sequence
  Primer Nsp1R
ccgctcgaggccgtaccacttgtgac

SEQ ID NO: 30 - DNA, 26 bp, Artificial Sequence
  Primer Nsp2pF
cgcggatccgctggaaagagagcaag

SEQ ID NO: 31 - DNA, 33 bp, Artificial Sequence
  Primer Nsp2pR
ccgctcgagtcgagtatcatttttgggaggaac

SEQ ID NO: 32 - DNA, 32 bp, Artificial Sequence
  Primer Nsp4F
cgcggatccggtgctttcagaactcgaaagcc

SEQ ID NO: 33 - DNA, 30 bp, Artificial Sequence
  Primer Nsp4R
ccgctcgagttccagttcaggtttggcagc

SEQ ID NO: 34 - DNA, 33 bp, Artificial Sequence
  Primer Na-nsp7F
cgcggatcctctctgactggtgccctcgctatg

SEQ ID NO: 35 - DNA, 29 bp, Artificial Sequence
  Primer Na-nsp7R
ccgctcgagttcccattgaactcttccat

SEQ ID NO: 36 - DNA, 30 bp, Artificial Sequence
  Primer Eu-nsp7F
ggaattctccctgacggctgctctagcttg

SEQ ID NO: 37 - DNA, 33 bp, Artificial Sequence
  Primer Eu-nsp7R
gtgctcgagtttcaaggcagttgtcaggcttgg

SEQ ID NO: 38 - DNA, 28 bp, Artificial Sequence
  Primer Nsp8F
cgcggatccgctgcaaagctttccgtgg

SEQ ID NO: 39 - DNA, 31 bp, Artificial Sequence
  Primer Nsp8R
ccgctcgaggtttaaacactgctccttagtc

SEQ ID NO: 40 - DNA, 807 bp, Porcine reproductive and respiratory syndrome virus
tccctgacggctgctctggcttgcaagttgtcgcaagctgaccttgactttttgtccagcttaacgaacttcaagtgctttgtatctgcctcaaacatgaaaaatgccgccggccagtacattgaggcagcttatgctagggccctacgccaggagttggcttctctggttcaggttgataaaatgaaagggattctatccaagcttgaggcctttgctgaaacggccactccgtcccttgacacaggtgacgtagttgttctacttgggcaacatcctcgcggatccatcctcgatattaatgtagggactgaaaggaaaactgtgtctgtgcaagagacccggagcttaggtggttccaaattcagtgtttgcactgtcgtgtccaacacacccgtggacgccttaactggcatcccactccagacaccaacccctctttttgagaatggcccgcgtcatcgtggtgaggaagacgaccttagtgtcgagaggaagaagaaacactgtgtgtccctcggcttccacaacattaatggcaaagtttactgcaaaatttgggacaagtccaccggagataccttttataccgatgattcccggtacacccaagaccttgcattccaggacaggtcagccgactacagagacagggattatgagggtgtgcaaaccgccccccaacagggctttgatccaaagtctgaaacccctattggcactgtagtgatcggcggtgttacgtataacaggtacctgatcaaaggtaaggaggtcttggttcccaagcctgacaactgccttgaa

SEQ ID NO: 41 - DNA, 807 bp, Porcine reproductive and respiratory syndrome virus
tccctgacggctgctctagcttgcaagttgtcgcaggctgatcttgacttcttgtccagcttgacgaacttcaagtgctttgtatctgcttcaaacatgaaaaatgctgctggccagtacattgaagcagcttatgctaaggccctgcgccaagagttggcttctctggttcaggttgacaaaatgaaaggaattttgtccaagctcgaggcctttgctgaaacagccaccccgtcccttgacacaggtgacgtggttgttctgcttgggcaacatcctcacggatccatcctcgatattaatgtggggactgaaaggaaaactgtgtccgtgcaagagacccggagcctaggtggttccaaattcagtgtttgcactgtcgtgtccaacacacccgtggacgccttaactggcatcccactccagataccggcacctctttttgagaatggtccgcgtcatcgtggcgaggaagatgatcttaaagtcgagaggatgaagaaacactgtgtgtctctcggcttccacaacatcaatggcaaagtttactgtaaaatttgggacaagtctaccggtgacacattttacaccgacgattcccggtacacccaagatcatgcttttcaggacaggtcagctgactacagagacagggactatgagggtatgcgaaccgccccccaacagggatttgatccaaagtctgaaacccctgttggcactgttgtgatcggcggtattacgtataacaggtatctgatcaaaggtaaggaggtcctggttcccaagcctgacaactgccttgaa

SEQ ID NO: 42 - protein, 259 aa, Porcine; identical to SEQ ID NO: 13
  ref <308>: DATABASE ACCESSION NUMBER: NP_740601
  ref <309>: DATABASE ENTRY DATE: 1998-04-02
  ref <313>: RELEVANT RESIDUES IN SEQ ID NO: (1)..(259)

SEQ ID NO: 43 - protein, 269 aa, Porcine reproductive and respiratory syndrome virus
SLTAALACKLSQADLDFLSSLTNFKCFVSASNMKNAAGQYIEAAYAKALRQELASLVQVDKMKGILSKLEAFAETATPSLDAGDVVVLLGQHPHGSILDINVGTERKTVSVQETRSLGGSKFSVCTVVSNTPVDALTGIPLQTPTPLFENGPRHRGEEDDLRVERMKKHCVSLGFHNINGKVYCKIWDKSTGDTFYTDDSRYTQDLAFQDRSADYRDRDYEGVQTAPQQGFDPKSETPIGTVVIGGITYNRYLIKGKEVLVPKPDNCLE